(19) United States
(12) Patent Application Publication
(10) Pub. No.: US 2005/0085419 A1
(43) Pub. Date: Apr. 21, 2005

(54) Anti-growth factor receptor avidin fusion proteins as universal vectors for drug delivery

(75) Inventors: Sherie L. Morrison, Los Angeles, CA (US); William M. Pardridge, Pacific Palisades, CA (US); Manuel L. Penichet, Los Angeles, CA (US); Josefina M. Coloma, Albany, CA (US); Shin Seung-Uon, Los Angeles, CA (US); Patrick P. Ng, Los Angeles, CA (US)

Correspondence Address: DAVID J. OLDENKAMP, ESQ., SHAPIRO & DUPONT LLP, 233 WILSHIRE BOULEVARD, SUITE 700, SANTA MONICA, CA, 90401, US

(73) Assignee: The Regents of the University of California

(21) Appl. No.: 10/858,729

(22) Filed: Jun. 2, 2004

Related U.S. Application Data
(63) Continuation of application No. 09/621,202, filed on Jul. 21, 2000, now abandoned.
(60) Provisional application No. 60/145,552, filed on Jul. 23, 1999.

(51) Int. Cl. A61K 38/17; C07K 14/72

(52) U.S. Cl. 514/12; 530/350

(57) ABSTRACT

A fusion protein for delivery of a wide variety of agents to a cell via antibody-receptor-mediated endocytosis comprises a first segment and a second segment: the first segment comprising a variable region of an antibody that recognizes an antigen on the surface of a cell that after binding to the variable region of the antibody undergoes antibody-receptor-mediated endocytosis, and, optionally, further comprises at least one domain of a constant region of an antibody; and the second segment comprising a protein domain selected from the group consisting of avidin, an avidin mutein, a chemically modified avidin derivative, streptavidin, a streptavidin mutein, and a chemically modified streptavidin derivative. Typically, the antigen is a protein. Typically, the protein antigen on the surface of the cell is a receptor such as a transferrin receptor-or an insulin receptor. The invention also includes an antibody construct incorporating the fusion protein that is either a heavy chain or a light chain together with a complementary light chain or heavy chain to form an intact antibody molecule. The invention further includes targeting methods and screening methods.

### CROSS-REFERENCES

This application claims priority from Provisional Application No. 60/145,552 by S. L. Morrison et al., filed Jul. 23, 1999, and entitled “Anti-Growth Factor Receptor Avidin Fusion Proteins as Universal Vectors for Drug Delivery, which is incorporated herein by this reference.

### BACKGROUND OF THE INVENTION

This invention is directed to fusion proteins that incorporate avidin or an avidin analogue or derivative to as well as a protein domain that can bind a receptor on the surface of a target cell, as well as methods of preparation and use of such fusion proteins.

Efficient and specific targeting of an active agent to the desired site is a critical factor for the successful diagnosis and/or therapy of many diseases. One region of the body particularly difficult to target is the brain due to the presence of the high resistance blood-brain barrier (BBB) formed by tightly joined capillary endothelial cell membranes (1, 2, 3, 4 5). (References referred to by numerals in parentheses are those given in Example 1 below). The BBB effectively restricts transport from the blood of certain molecules, especially those that are water soluble and larger than several hundred daltons (6). In fact the clinical utility of many proteins of therapeutic interest for the brain is limited by their inability to cross the BBB. In some cases neurotrophic factors have been administered to the brain by invasive neurosurgical procedures (7, 8, 9) or grafting neurotrophin-producing cells into brain sites (10, 11). However, such surgical procedures are complex, carry risks of complications such as infection and damage to critical central nervous system structures, and are frightening to potential patients, who might fear the surgery to such an extent that they refuse to undergo it even when they could potentially benefit from it.

The BBB has been shown to have specific receptors which allow the transport from the blood to the brain of several macromolecules including insulin (12), transferrin (Tf) with iron attached (13), and insulin-like growth factors 1 and 2 (IGF1 and IGF2) (12, 14). Therefore, one noninvasive approach for the delivery of drugs to the brain is to attach the agent of interest to a molecule with receptors on the BBB which would then serve as a vector for transport of the agent across the BBB (15, 16). An alternative approach is the delivery of agents attached to an antibody (Ab) specific for one of the BBB receptors. Indeed, both nerve growth factor (NGF) and CD4 will cross the BBB when chemically conjugated to an Ab directed against the transferrin receptor CTfR) (17, 18, 19). Therefore, despite the fact that Abs normally are excluded from the brain (20), they can be an effective vehicle for the delivery of molecules into the brain parenchyma if they have specificity for receptors on the BBB. In fact, the intravenous injection of an anti-rat TfR AbNGF chemical conjugate prevented the loss of striatal choline acetyltransferase-immunoreactive neurons in a rat model of Huntington's disease and reversed the age-related cognitive dysfunction (21, 22). Recently, a fusion protein with NGF attached to the N-terminus of an Ab directed against human TfR using genetic engineering techniques (23) showed both antigen binding and NGF activity suggesting its therapeutic utility. Although promising, this approach that requires that unique chimeric molecules be constructed for each specific application, is cumbersome and sometimes can lead to the decrease or loss of activity of one or both of the covalently conjugated partners. To overcome these limitations it is therefore desirable to develop a universal delivery system that eliminates the need to make a specific construct for each individual application.

The ideal brain delivery vector should be able to deliver many different compounds which are bound to the vector by high affinity noncovalent interactions such as those seen between avidin (Av) and biotin. Indeed Ab-Av chemical conjugates have been used to deliver a mono-biotinylated drug (24, 25). However, an important drawback of the chemical coupling procedure is the difficulty in producing a reproducible, homogeneous product. The existence of impurities is particularly significant in a product intended to interact with the central nervous system. For example, several years ago, the United States Food and Drug Administration forced the amino acid tryptophan off the market as a nutritional supplement because of serious neurological side effects that occurred as the result of a trace contaminant formed during the fermentation process used to produce the amino acid.

There is also a need to target other cell types in other organs and organ systems. For example, there is a particular need to target liver cells to treat hepatitis and to target cancer cells.

Receptor-mediated endocytosis represents a highly efficient internalization pathway of eukaryotic cells and has been explored as a novel approach for nonviral delivery of gene therapy. To accomplish gene transfer a vector must contain two functional domains: a domain binding to receptors, and a DNA-binding domain that achieves interaction with the gene to be transported in a reversible, noncovalent, and nondamaging manner. Specific gene transfer can be achieved by Abs directed against specific receptors. Anti-receptor-based strategies for accomplishing gene transfer via receptor-mediated endocytosis have been successful in squamous carcinoma cells using a monoclonal Ab directed against the receptors for erythrocyte growth factor chemically conjugated to polylysine to deliver DNA associated with the polylysine (26, 27). Alternatively, natural ligands, such as polylysine conjugated to EGF, have been used for gene delivery via receptor-mediated endocytosis (26, 28). Importantly, Tf conjugated with protamine or polylysine molecules has also been used for high-efficiency delivery of double-stranded DNA, single-stranded DNA, and modified RNA molecules independent of nucleic acid size (from short oligonucleotides to DNA of 21 kilobase pairs), a procedure called “transferr infection” (29, 30). Biotinylated double-stranded DNA conjugated to biotinylated Tf via streptavidin was successfully transduced into TfR-positive human cancer cells (31). More recently, a biotinylated recombinant adenovirus vector bound to the biotinylated ligand for the c-Kit receptor stem cell factor (SCF) through an avidin bridge showed a notable increase in cell targeting and gene expression (32). These studies validate the concept that gene delivery may be achieved via Ab-based or ligand-based targeting of the receptor-mediated endocytotic pathway, although improved methods of exploiting this pathway are required.

Despite the considerable advancement in anti-cancer therapy, minimal residual disease is still a major problem in the clinical management of cancer. Chemotherapeutic strategies are necessarily limited by various toxicities, and of limited efficacy against nonproliferating tumor cells. Additional modalities, which will achieve further cytoreduction are needed. One approach to this problem has been to use Abs or growth factors as targeting vehicles for the specific delivery of cytotoxic molecules to target cells. Initially this was achieved by chemically conjugating the two moieties, however the use of chemical conjugates has many drawbacks including a lack of homogeneity. An alternative approach has been to genetically fuse the two moieties. The bacterial toxins diphtheria toxin (DT) and Pseudomonas exotoxin A (PE) (33), the plant toxin ricin (33) and mammalian ribonuclease A (RNase A) (34, 35) have been used successfully as cytotoxic agents in this approach. All of these molecules rely on internalization and intracellular processing to exert their cytotoxic effects and cell surface receptors such as the IL-2R and TfR have been exploited to achieve this. Significant advances have been made in the development of such targeted therapies and fusion proteins have shown anti-tumor efficacy leading to clinical trials (36, 37). However, a limitation of this approach is that it requires that specific fusion proteins be constructed for each specific application, which is cumbersome and sometimes can lead to the decrease or loss of activity of one or both covalently conjugated partners.

It is therefore desirable to develop a universal delivery system that eliminates the need to make a specific construct for each individual application. To achieve this, I propose the development of novel universal vectors which will make it possible to target a broad range of anti-cancer agents to tumor cells expressing the IL-2R and/or TfR. These vectors will make it much easier to treat tumors with multiple different anticancer agents and should result in more effective anti-tumor activity with fewer toxic side-effects. The proposed vectors can serve not only to carry proteins or chemical compounds, but also to carry DNA of a wide range of sizes allowing specific and effective in vitro and in vivo gene transfer into the tumor cells. The vectors should thus be superior to the currently used retrovirus and adenovirus vectors which are hampered by limits of the size of the genetic material to be transferred, potential safety problems and limited specific targeting in vivo (31). It should be noted that the proposed universal vectors should be able to specifically target dansylated or biotinylated viruses in a manner similar to what was recently reported for a biotinylated recombinant adenovirus vector bound to the biotinylated ligand for the c-Kit receptor stem cell factor (SCF) through an avidin bridge (32).

The primary function of serum transferrin (Tf) is to bind iron and tnansport it through the blood (38). Tf donates iron to cells through its interaction with the transferrin receptor (TfR) (38). After binding to its receptor on the cell surface, Tf is internalized into an acidic compartment where iron dissociates and the apo-Tf is returned to the cell surface where ligand-receptor dissociation occurs. It has also been proposed that the TfR serves a role as a growth factor independent of its function as a transporter of iron (39). Tf is considered to be an autocrine regulator of cell proliferation in malignant tumor cells (39). High level expression of the TfR has been identified on many malignant tumors such as lymphomas (40) and leukemias (41). Like the IL-2R, constitutive expression of the TfR is not limited to hematopoietic neoplasms, but has been detected in other kinds of malignant tumors such as gastric cancer (42), uterine cancer (43), breast cancer (44), and bladder cancer (45). Therefore the TfR expressed on tumor cells should be a suitable target for the delivery of cytotoxic drugs into the cancer cells by receptor mediated endocytosis.

Therefore, there is a need for an improved method of targeting particular cell types with a vector that is suitable for use with a large variety of compounds to be targeted and that need not be developed from scratch for each compound. There is also a need for an improved method of targeting that avoids the use of chemical conjugation with its side reactions and production of contaminants. There is a particular need for improved targeting methods for the central nervous system and for other cell types, such as liver cells and malignant cells.

### SUMMARY

We have developed fusion proteins and antibody construct that can be used to target biotin-linked compounds to cells. After binding to the surface, the fusion protein or antibody construct and its attached cargo undergo antibody-receptor-mediated endocytosis.

One embodiment of the present invention is a fusion protein comprising a first segment and a second segment:
(1) the first segment comprising a variable region of an antibody that recognizes an antigen on the surface of a cell that after binding to the variable region of the antibody undergoes antibody-receptor-mediated endocytosis, and, optionally, further comprises at least one domain of a constant region of an antibody; and
(2) the second segment comprising a protein domain selected from the group consisting of avidin, an avidin mutein, a chemically modified avidin derivative, streptavidin, a streptavidin mutein, and a chemically modified streptavidin derivative.

The antigen on the surface of the cell can be, but is not limited to, a protein.

Typically, when the antigen is a protein, the protein on the surface of the cell is a receptor. The receptor can be a growth factor receptor, such as epidermal growth factor receptor, vascular endothelial growth factor receptor, an insulin-like growth factor receptor, platelet-derived growth factor receptor, transforming growth factor β receptor, fibroblast growth factor receptor, interleukin-2 receptor, interleukin-3 receptor, erythropoietin receptor, nerve growth factor receptor, brain-derived neurotrophic factor receptor, neurotrophin-3 receptor, and neurotrophin-4 receptor. Alternatively, the receptor can be transferrin receptor or insulin receptor.

Typically, the second section of the fusion protein comprises avidin.

The antigen can be an antigen on the surface of a human cell or on the surface of a cell of another socially or economically important mammal such as a dog, a cat, a horse, a cow, a pig, or a sheep. If the antigen is on the surface of a human cell, it can be, but is not limited to, the human transferrin receptor or the human insulin receptor.

When the fusion protein comprises at least one domain of a constant region of an antibody, various alternative arrangements are possible. These include but are not limited to the following. For example, in one arrangement, the entire constant region of the heavy chain is present and the second segment is located to the carboxyl-terminal side of the C_{H}3 region in the fusion protein. In another arrangement, the C_{H}1 and hinge region domains are present and the second segment is located to the carboxyl-terminal side of the hinge region in the fusion protein. In yet another arrangement, the C_{H}1 domain is present and the second segment is located to the carboxyl-terminal side of the C_{H}1 domain in the fusion protein. Alternatively, the constant region of the light chain is present and the second segment is located to the carboxyl-terminal side of C_{L′} in the fusion protein.

The fusion protein can be a single-chain antibody molecule (sFv).

Another embodiment of the present invention is an antibody construct comprising:
(1) two fusion protein chains, each comprising a first segment and a second segment:
   (a) the first segment comprising a variable region of an antibody that recognizes an antigen on the surface of the cell that after binding to the variable region of the antibody undergoes antibody-receptor-mediated endocytosis, and a constant region of an antibody; and
   (b) the second segment comprising a protein domain selected from the group consisting of avidin, an avidin mutein, a chemically modified avidin derivative, streptavidin, a streptavidin mutein, and a chemically modified streptavidin derivative; wherein the fusion protein chains comprise either light chains or heavy chains of an antibody molecule; and
(2) two chains of an antibody molecule that are either heavy chains, if the fusion protein chains of (a) are light chains, or are light chains, if the fusion protein chains of (a) are heavy chains; wherein the light chains and heavy chains are assembled by noncovalent interactions and disulfide bonds.

The antibody construct is therefore a complete antibody molecule, with two heavy chains and two light chains, but including avidin or streptavidin or a mutein or derivative as discussed above.

Another embodiment of the present invention is a method for targeting a compound to a cell surface comprising the steps of:
(1) linking the compound to biotin or a biotin analogue to form a conjugate recognized by avidin or streptavidin or their derivatives;
(2) binding the conjugate to a fusion protein or to an antibody construct according to the present invention; and
(3) binding the fusion protein or antibody construct bound to the conjugate to target the compound to the cell surface.

The cell to be targeted can be any cell bearing a surface receptor recognized by the antibody. Possible target cells include, but are not limited to, a liver cell, a malignant cell, a cell that is a component of the central nervous system, or an endothelial cell of the blood-brain barrier.

The compound to be targeted can be a protein, a nucleic acid, or another compound. For example, the compound can be a radioactively labeled organic or inorganic molecule. If the compound is a nucleic acid, it can be a gene expression vector or an RNA. The compound can also be a peptide nucleic acid. If the compound is a nucleic acid or a peptide nucleic acid, it can have antisense activity. One particular peptide nucleic acid that can be targeted is a peptide nucleic acid of the structure 5′-biotin-CTCCGCTTCTTCCTGCCA-Tyr-Lys-CONH₂-3′. This peptide nucleic acid can be targeted to the brain.

Another embodiment of the present invention is a screening method for determining the cytotoxicity of a compound comprising the steps of:
(1) linking the compound to biotin or a biotin analogue to form a conjugate recognized by avidin or streptavidin or their derivatives;
(2) binding the conjugate to a fusion protein or to an antibody construct of the present invention;
(3) binding the fusion protein or antibody construct bound to the conjugate to the surface of a cell in which cytotoxicity is to be screened;
(4) allowing the biotin conjugate bound to the fusion protein to undergo antibody-receptor-mediated endocytosis; and
(5) determining the cytotoxicity of a compound by determining the survival of cells penetrated by the compound with the survival of a control sample of cells to which the fusion protein or antibody construct bound to the biotin conjugate has not been targeted to determine the cytotoxic effect of the compound upon endocytosis.

The cell can be a liver cell, a cell that is a component of the central nervous system, or a malignant cell. The compound for which cytotoxicity is being screened can be as described above.

Another embodiment of the present invention is a nucleic acid molecule encoding a fusion protein of the present invention. Typically, the nucleic acid molecule is DNA.

Another embodiment of the present invention is a vector comprising the DNA operably linked to at least one control element that effects the transcription, translation, or replication of the DNA.

Still another embodiment of the present invention is a host cell transfected with the vector.

Another embodiment of the present invention is a method for producing a purified fusion protein comprising the steps of:
(1) culturing the host cell transfected with the vector under conditions in which the fusion protein is synthesized; and
(2) purifying the synthesized fusion protein from the cultured host cell or from culture medium in which the host cell has been cultured to produce purified fusion protein.

Another embodiment of the present invention is a nucleic acid array comprising:
(1) a nucleic acid molecule encoding a fusion protein comprising a first segment and a second segment:
   (a) the first segment comprising a variable region of an antibody that recognizes a protein on the surface of the cell that after binding to the variable region of the antibody undergoes antibody-receptor-mediated endocytosis, and a constant region of an antibody; and
   (b) the second segment comprising a protein domain selected from the group consisting of avidin an avidin mutein, a chemically modified avidin derivative, streptavidin, a streptavidin mutein, and a chemically modified streptavidin derivative; wherein the fusion protein comprises either a light chain or a heavy chain of an antibody molecule; and
(2) a nucleic acid molecule encoding an antibody chain complementary to the antibody chain encoded by the nucleic acid of (a), wherein when the nucleic acid molecule of (a) encodes a light chain, the nucleic acid molecule of (b) encodes a heavy chain, and wherein when the nucleic acid molecule of (a) encodes a heavy chain, the nucleic acid molecule of (b) encodes a light chain.

Yet another embodiment of the present invention is a method for producing a purified antibody construct comprising:
(1) transfecting a host cell with a vector including a nucleic acid molecule encoding a fusion protein comprising a first segment and a second segment:
   (a) the first segment comprising a variable region of an antibody that recognizes a protein on the surface of the cell that after binding to the variable region of the antibody undergoes antibody-receptor-mediated endocytosis, and a constant region of an antibody; and
   (b) the second segment comprising a protein domain selected from the group consisting of avidin, an avidin mutein, a chemically modified avidin derivative, streptavidin, a streptavidin mutein, and a chemically modified streptavidin derivative; wherein the fusion protein comprises either a light chain or a heavy chain of an antibody molecule;
(2) transfecting the host cell transfected in step (1) with a vector including a nucleic acid molecule encoding an antibody chain complementary to the antibody chain encoded by the nucleic acid of (1), wherein when the nucleic acid molecule of (1) encodes a light chain, the nucleic acid molecule of (2) encodes a heavy chain, and wherein when the nucleic acid molecule of (1) encodes a heavy chain, the nucleic acid molecule of (2) encodes a light chain;
(3) culturing the host cell after the transfection of step (2) under conditions in which the antibody construct is synthesized; and
(4) purifying the synthesized antibody construct from the cultured host cell or from culture medium in which the host cell has been cultured to produce purified antibody construct.

As an alternative for producing the antibody construct, the heavy chain and light chain can be assembled after synthesis in separate cells. This method comprises:
(1) tnansfecting a first host cell with a vector including a nucleic acid molecule encoding a fusion protein comprising a first segment and a second segment:
   (a) the first segment comprising a variable region of an antibody that recognizes a protein on the surface of the cell that after binding to the variable region of the antibody undergoes antibody-receptor-mediated endocytosis, and a constant region of an antibody; and
   (b) the second segment comprising a protein domain selected from the group consisting of avidin, an avidin mutein, a chemically modified avidin derivative, streptavidin, a streptavidin mutein, and a chemically modified streptavidin derivative; wherein the fusion protein comprises either a light chain or a heavy chain of an antibody molecule;
(2) transfecting a second host cell with a vector including a nucleic acid molecule encoding an antibody chain complementary to the antibody chain encoded by the nucleic acid of (1), wherein when the nucleic acid molecule of (1) encodes a light chain, the nucleic acid molecule of (2) encodes a heavy chain, and wherein when the nucleic acid molecule of (1) encodes a heavy chain, the nucleic acid molecule of (2) encodes a light chain;
(3) culturing the host cells transfected in steps (1) and (2) under conditions in which the fusion protein of (1) and the antibody chain of (2) are synthesized:
(4) purifying the fusion protein of (i) and the antibody chin of (2) from the cultured host cells or from culture media in which the host cells have been cultured to produce a purified fusion protein and a purified antibody chain; and
(5) assembling the fusion protein of (1) and the antibody chain of (2) to produce a purified antibody construct.

### BRIEF DESCRIPTION OF THE DRAWINGS

The following invention will become better understood with reference to the specification, appended claims, and accompanying drawings, where:

FIG. 1 is a schematic diagram of the construction and expression of the antibody construct of the Example;

FIG. 2 is an electropherogram of SDS-PAGE analysis of the antibody construct of the Example under non-reducing (A) and reducing (B) conditions;

FIG. 3 is a graph of flow cytometric results demonstrating the specificity of the antibody construct of the Example for the transferrin receptor expressed on the surface of cultured rat cells: (A) negative control antibody; (B) positive control antibody; and (C) antibody construct;

FIG. 4 is a graph of immunoassay results showing the binding of the antibody construct of the Example to biotinylated BSA coated microtiter plates: (A) antibody construct was added at varying concentrations with/without previous incubation with biotin acrylic beads and the bound protein detected using anti-kappa conjugated with alkaline phosphatase. (B) antibody construct (2.5 nM) preincubated with varying concentrations of biotinylated BSA was added to the biotinylated BSA coated microtiter plates and bound Ab detected using anti-kappa conjugated with alkaline phosphatase;

FIG. 5 is a graph showing plasma clearance of proteins: the plasma profiles of ¹²⁵I-OX-26 and of [³H]-biotin bound to either the OX-26/Av conjugate, or anti-TfR IgG3-C_{H}3-Av (the antibody construct of the Example) fusion protein were analyzed; the open triangles represent ¹²⁵I-OX-26, the open circles anti-TfR OX-26/Av conjugate, the filled circles anti-TfR IgG3-C_{H}3-Av: % ID/ml represents percentage of injected dose per ml plasma;

FIG. 6 is a graph that shows that anti-rat TfK IgG3-C_{H}3-AV bind the Ton the surface of Y3-Ag1.2.3 as detected by flow cytometry; the extent of TfR binding by anti-rat TfR IgG3-C_{H}3-Av was similar to that observed with anti-rat TfR IgG3 (data not shown), suggesting that the antibody keeps intact its antigen recognition ability after its fusion to the avidin;

FIG. 7 is a graph showing that that with both complexes: (anti-rat TfR IgG3-C_{H}3-Av)-(biotinylated β-gal) and (anti-rat TfR IgG3-C_{H}3-Av)-(biotinylated DNA) are able to target the TfR on the surface of Y3-Ag1.2.3; and

FIG. 8 is a graph showing that the universal vector anti-rat TfR IgG3-C_{H}3-Av can be used to deliver biotinylated β-gal enzyme as well as biotinylated plasmid encoding for β-gal (pCH 104) into Y3-Ag1.2.3 cells.

### DEFINITIONS

As used herein, the terms defined below have the following meanings unless otherwise indicated:

“Nucleic Acid”: the term “nucleic acid” includes both DNA and RNA unless otherwise specified, and, unless otherwise specified, includes both double-stranded and single-stranded nucleic acids. If a single-stranded nucleic acid is recited, the recitation also includes the complement according to Watson-Crick base pairing rules unless the complement is excluded. Also included are hybrids such as DNA-RNA hybrids. In particular, a reference to DNA includes RNA that has either the equivalent base sequence except for the substitution of uracil and RNA for thymine in DNA, or has a complementary base sequence except for the substitution of uracil for thymine, complementarity being determined according to the Watson-Crick base pairing rules. Reference to nucleic acid sequences can also include modified bases as long as the modifications do not significantly interfere either with binding of a ligand such as a protein by the nucleic acid or with Watson-Crick base pairing.

“Antibody”: as used herein the term “antibody” includes both intact antibody molecules of the appropriate specificity, and antibody fragments (including Fab, F(ab′), Fv, and F(ab′)₂), as well as chemically modified intact antibody molecules and antibody fragments, including hybrid antibodies assembled by in vitro reassociation of subunits. Also included are single-chain antibody molecules generally denoted by the term sFv and humanized antibodies in which some or all of the originally non-human constant regions are replaced with constant regions originally derived from human antibody sequences. Both polyclonal and monoclonal antibodies are included unless otherwise specified. Additionally included are modified antibodies or antibodies conjugated to labels or other molecules that do not block or alter the binding capacity of the antibody.

### DESCRIPTION

We have developed fusion proteins that incorporate both a binding segment for a molecule on the surface of a cell to be targeted and an avidin or avidin analogue. This allows the cell to be targeted by binding the molecule to be targeted to biotin and then binding the conjugate of biotin and the molecule to be targeted to the fusion protein. This allows the use of the specificity and high affinity of the biotin-avidin link to target any molecule that can be linked to biotin.

In the case of brain targeting, the complex of the molecule to be targeted and the fusion protein of the invention will bind to the blood-brain barrier (BBB) receptors which are present on the luminal membrane of brain capillary endothelial cells. Through the process of receptor-mediated endocytosis, the fusion protein is internalized into vesicular structures within the endothelial cells. Then, the fusion protein is transported to and released from the abluminal surface of the capillary endothelial cell and, once released into the brain, diffuses into the parenchyma The whole process is known as transcytosis.

When targeting a surface receptor on cells that are not on the BBB, the fusion protein is internalized into vascular structures within the cell. If the cargo molecule is a protein, it can now function within the cell. If the cargo molecule is a gene, it can be expressed. The uptake process is known as endocytosis. This approached can be used for the diagnosis and/or treatment of a broad range of liquid and solid tumors which express the TfR and/or the IR. For example, specific delivery of radioactive compounds, enzymes, or toxins to cancer cells and specific delivery of genes to cancer cells (gene therapy) can be performed. The utility of the universal delivery system is not restricted to the elimination of tumor cells in vivo but can also be used for in vitro approaches including the efficient purging of cancer cells during ex vivo expansion of hematopoietic progenitor cells for use as an autograft. It can also be used to target and treat receptor bearing cells in the liver.

I. Fusion Proteins Incorporating Avidin and a Binding Segment

One aspect of the present invention is a fusion protein that incorporates both a binding segment and avidin.

In general, a fusion protein according to the present invention comprises: a fusion protein comprising a first segment and a second segment:
(1) the first segment comprising a variable region of an antibody that recognizes an antigen on the surface of a cell that after binding to the variable region of the antibody undergoes antibody-receptor-mediated endocytosis, and, optionally, further comprising at least one domain of a constant region of an antibody; and
(2) the second segment comprising a protein domain selected from the group consisting of avidin, an avidin mutein, a chemically modified avidin derivative, streptavidin, a streptavidin mutein, and a chemically modified streptavidin derivative.

The antigen on the surface of the cell can be, but is not limited to, a protein. Alternatively, it can be a nonprotein antigen.

For the first segment, the antigen on the surface of the cell is typically a receptor. In one particularly preferred embodiment, the receptor can be a growth factor receptor, such as, but not limited to, epidermal growth factor, vascular endothelial growth factor, an insulin-like growth factor, platelet-derived growth factor, transforming growth factor A, fibroblast growth factor, interleukin-2, interleukin-3, erythropoietin, nerve growth factor, brain-derived neurotrophic factor, neurotrophin-3, and neurotrophin-4. In another particularly preferred embodiment, the receptor can be a receptor selected from the group consisting of transferrin receptor and insulin receptor.

The antigen can be an antigen on the surface of a human cell or on the surface of a cell of another socially or economically important mammal such as a dog, a cat, a horse, a cow, a pig, or a sheep. If the antigen is on the surface of a human cell, it can be, but is not limited to, the human transferrin receptor or the human insulin receptor.

The second segment can be avidin, a chemically modified avidin derivative, or a avidin mutein in which the amino acid sequence of the avidin is altered by genetic engineering techniques such as site-specific mutagenesis, for example to remove cysteine residues. Alternatively, the bacterial avidin analogue streptavidin can be used in place of avidin, so that the second segment can be streptavidin, a chemically modified streptavidin derivative, or a streptavidin mutein. Typically, the second segment is avidin.

Optionally, but preferably, the first segment further includes at least one domain of a constant region of an antibody. Various arrangements are possible. These include but are not limited to the following. For example, in one arrangement, the entire constant region of the heavy chain is present and the second segment is located to the carboxyl-terminal side of the C_{H}3 region in the fusion protein. In another arrangement, the C_{H}1 and hinge region domains are present and the second segment is located to the carboxyl-terminal side of the hinge region in the fusion protein. In yet another arrangement, the C_{H}1 domain is present and the second segment is located to the carboxyl-terminal side of the C_{H}1 domain in the fusion protein. Alternatively, the constant region of the light chain is present and the second segment is located to the carboxyl-terminal side of C_{L′} in the fusion protein.

In another alternative, the first segment includes domains derived from the heavy and light chain of an antibody molecule, including the variable regions and a sufficient portion of the constant regions, joined by linkers, so that the entire fusion protein forms a single-chain antibody (sFv). Single-chain antibodies are described, for example, in C. A. K. Borrebaeck, ed., “Antibody Engineering” (2d ed., Oxford University Press, New York, 1995).

The fusion protein can further include linkers positioned either between the first and second segments or within the first segment to ensure that both segments of the resulting fusion protein retain their desired binding activity. The linkers are typically 3 to 25 amino acids in length. Typically, the amino acids within the linkers are aliphatic, although other amino acids, such as uncharged polar amino acids, can also be included. Typically, the linkers form α-helices, although linkers that form random coils can also be used.

Immunoglobulin fusion proteins and analogues are described, for example, in U.S. Pat. No. 5,844,095 to Linsley et al., U.S. Pat. No. 5,968,510 to Linsley et al., U.S. Pat. No. 5,977,318 to Linsley et al., U.S. Pat. No. 5,637,481 to Ledbetter et al., U.S. Pat. No. 5,521,288 to Linsley et al., U.S. Pat. No. 5,428,130 to Capon et al., and U.S. Pat. No. 5,116,964 to Capon et al., all of which are incorporated herein by this reference.

Antibody-avidin fusion proteins are described in S.-U. Shin et al., “Functional and Pharmacokinetic Properties of Antibody-Avidin Fusion Proteins,” J. Immunol. 158: 4797-4804 (1997), incorporated herein by this reference, as well as in P. P. Ng, J. S. Dela Cruz, S.-U. Shin, S. L. Morrison, and M. L. Penichet, “Characterization of an Antibody-Avidin Fusion Protein Specific for the Transferrin Receptor as Gene Delivery Vehicle into Cancer Cells” (presented at 91 st Annual Meeting of the American Association for Cancer Research, San Francisco, Calif., USA, Apr. 1-5, 2000), Proc. Am. Assoc. Cancer Res. 41: 451 (2000).

II. Antibody Constructs

Another aspect of the present invention is an antibody construct incorporating the first and second segments of the fusion protein in a complete, intact antibody molecule. In general, an antibody construct according to the present invention comprises:
(1) two fusion protein chains, each comprising a first segment and a second segment:
   (a) the first segment comprising a variable region of an antibody that recognizes an antigen on the surface of the cell that after binding to the variable region of the antibody undergoes antibody-receptor-mediated endocytosis, and a constant region of an antibody; and
   (b) the second segment comprising a protein domain selected from the group consisting of avidin, an avidin mutein, a chemically modified avidin derivative, streptavidin, a streptavidin mutein, and a chemically modified streptavidin derivative; wherein the fusion proteins comprise either light chains or heavy chains of an antibody molecule; and
(2) two chains of an antibody molecule that are either heavy chains, if the fusion protein chains of (1) are light chains, or are light chains, if the fusion protein chains of (1) are heavy chains.

The light chains and heavy chains are assembled by noncovalent interactions and disulfide bonds.

The first and second segments are as described above. Typically, the antigen on the surface of the cell is a protein. If the antigen is a protein, typically, it is a receptor, as described above. The antigen can be an antigen on the surface of a human cell, as described above, or on the surface of a non-human cell. Typically, the protein domain of the second segment is avidin, as described above. In one preferred embodiment, the avidin is chicken avidin, but other avidins can also be used for the protein domain.

The antibody in the antibody construct can be human, non-human, humanized, or chimeric. The antibody can be of any isotype. In one preferred embodiment, the antibody is an IgG3 antibody.

III. Nucleic Acid Molecules

Another aspect of the present invention is a nucleic acid molecule that encodes a fusion protein of the present invention. The nucleic acid molecule is typically DNA.

Yet another aspect of the present invention is a vector comprising the DNA operably linked to at least one control element that affects the transcription, translation, or replication of the DNA.

Still another aspect of the present invention is a host cell transfected with the vector.

The control elements of the vector can be promoters, operators, enhancers, or other nucleic acid sequences that affect the transcription, translation, or replication of the DNA. The vector can be derived from either prokaryotic or eukaryotic sources. The vector can comprise sequences of chromosomal, non-chromosomal, or synthetic DNA sequences. Typically, these vectors include one or more cloning sites that contain restriction endonuclease sequences that are readily cleavable by specific restriction endonucleases. It is generally preferred that these restriction endonucleases yield-cohesive or “sticky” ends for more efficient cloning in of the desired sequence. Some suitable prokaryotic cloning vectors include plasmids from Escherichia coli, such as cotel, pCR1, pBR322, pMB9, pUC, pKSM, or RP4. Prokaryotic vectors also include derivatives of bacteriophage DNA such as M13 and other filamentous single-stranded DNA phages. Other vectors, such as baculovirus vectors, can be used.

Examples of useful expression control sequences are the lac system, the trp system, the tac system, the trc system, major operator and promoter regions of bacteriophage lambda, the control region of fd coat protein, the glycolytic promoters of yeast, e.g., the promoter for 3-phosphoglycerate kinase, the promoters of yeast acid phosphatase, e.g., Pho5, the promoters of the yeast alpha-mating factors, and promoters derived from polyoma, adenovirus, retrovirus, and simian virus, e.g., the early and late promoters of SV40 and other sequences known to control the expression of genes of prokaryotic or eukaryotic cells and their viruses or combinations thereof. Vectors useful in yeast are available. A suitable example is the 2μ plasmid. Vectors for use in animal cells are also known. These vectors include derivatives of SV40, adenovirus, retrovirus-derived DNA sequences, and shuttle vectors derived from combinations of functional mammalian vectors, such as those described above, and functional plasmids and phage DNA. Another suitable vector is the baculovirus vector.

Vectors are inserted into a host cell for expression. Typically, these vectors are inserted into a host cell by methods well-known in the art, such as transfection, transformation, electroporation, direct injection of the DNA, lipofection, and other well-understood methods. The method to be used can be chosen according to the host cells selected and the size and conformation of the DNA. Some useful expression host cells include well-known prokaryotic and eukaryotic cells. Some suitable prokaryotic hosts include, for example, E. coli, such as E. coli SG-936, E. coli HB101, E. coli W3110, E. coli Ω1776, E. coli Ω2282, E. coli DHI, and E. coli MRCI. Other bacterial and fungal host cells could be used, such as Pseudomonas, Bacillus species, such as Bacillus subtilis, and Streptomyces. Other host cells that can be used are eukaryotic cells such as yeast and other fungi, insect cells, animal cells, such as COS cells and CHO cells, human cells, and plant cells in tissue culture.

Cloning methods and expression methods are described, for example, in D. V. Goeddel, ed., “Gene Expression Technology” (Methods in Enzymology, vol. 185, Academic Press, San Diego, 1991), J. Sambrook et al., “Molecular Cloning: A Laboratory Manual” (Cold Spring Harbor Laboratory Press, 1989), and B. Perbal, “A Practical Guide to Molecular Cloning” (John Wiley & Sons, 1988).

Yet another aspect of the present invention is a nucleic acid array comprising:
(1) a nucleic acid molecule encoding the fusion protein that forms part of the antibody construct described above; and
(2) a nucleic acid molecule encoding an antibody chain complementary to the antibody chain encoded by the nucleic acid of (1), wherein when the nucleic acid molecule of (a) encodes a light chain, the nucleic acid molecule of (2) encodes a heavy chain, and wherein when the nucleic acid molecule of (1) encodes a heavy chain, the nucleic acid molecule of (2) encodes a light chain.

Preferably, the nucleic acid molecules of the nucleic acid array are DNA.

IV. Methods for Producing Fusion Proteins and Constructs

Methods for producing fusion proteins according to the present invention are well known in the art. In general, such methods comprise:
(1) culturing a host cell transformed with a vector including DNA encoding a fusion protein according to the present invention as described above; and
(2) purifying the synthesized fusion protein from the cultured host cells or from culture medium in which the host cells have been cultured to produce purified fusion protein.

Several methods can be used to produce antibody constructs according to the present invention. For example, nucleic acid molecules encoding each of the two chains can be incorporated into vectors and the vectors can then be used to transfect separate host cells for expression of the heavy chains and light chains. The resulting heavy chains and light chains can then be assembled in vitro under conditions that permit proper folding and formation of disulfide bonds. Such conditions are generally known in the art and are described, for example, in J. L. Cleland & C. S. Craik, eds., “Protein Folding” (Wiley-Liss, New York, 1996), ch. 10, pp. 283-298.

It is particularly preferred to produce antibody constructs according to the present invention in myeloma cells. Such cells efficiently glycosylate the constructs so that they have the desired activity. Chinese hamster ovary (CHO) cells provide an alternative mammalian expression system. Baculovirus is an example of a non-mammalian expression system.

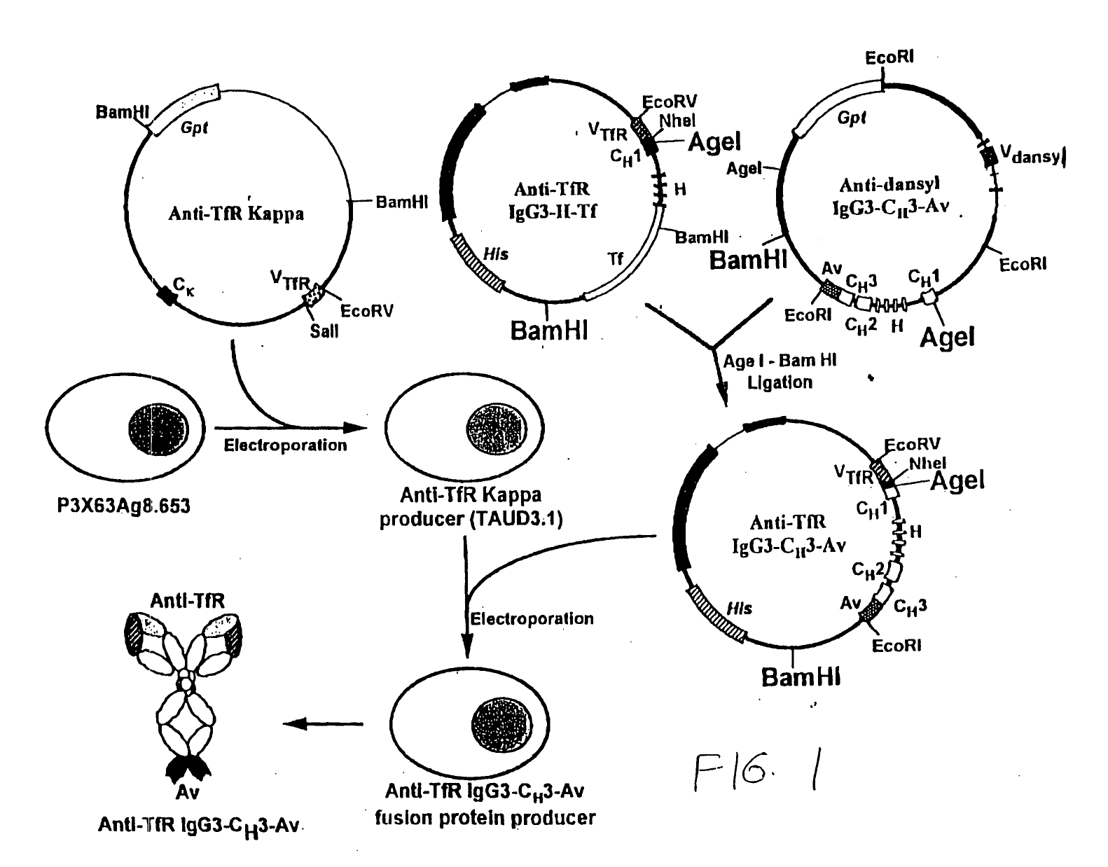

Alternatively, the vectors incorporating nucleic acids encoding both the heavy and light chains can be transfected into the same host cell, either simultaneously, or, more typically, sequentially, so that a transfectant for either the heavy chain or the light chain is used as a recipient for further transfection. The construct is then produced by expression of both heavy and light chains in the doubly transfected host cell. This approach is shown in FIG. 1 of the Example, below.

Expression methods are well known in the art and are described in the references above in Section III.

V. Targeting Methods

Another aspect of the present invention is a method for targeting a compound to a cell surface comprising the steps of:
(1) linking the compound to biotin or a biotin analogue to form a conjugate recognized by avidin or streptavidin or their derivatives;
(2) binding the conjugate to a fusion protein or antibody construct according to the present invention as described above; and
(3) binding the fusion protein or antibody construct bound to the conjugate to target the compound to the cell surface.

The compound to be targeted can be, but is not limited to, a protein or a nucleic acid. If the compound is a nucleic acid, the compound can be an antisense nucleic acid or an antisense nucleic acid analogue or derivative such as a peptide nucleic acid. Other antisense nucleic acid analogues are known in the art, such as phosphorothioates, phosphorodithioates, methylphosphonates, and 2′-O-methyloligoribonucleotides. Alternatively, if the compound is a nucleic acid, it can be a gene expression vector for expression of a desired product or an RNA. The compound can be a radioactively labeled organic or inorganic molecule. If the compound is a protein, it can be an enzyme, an antibody, a receptor, or any other protein with a specific biological activity. The compound can also be a radioactive compound, a drug, such as an antineoplastic drug, or a toxin.

Many methods are well known for linking compounds to biotin. Typically, the linkage is covalent, and a spacer can be included. Biotinylation reagents and methods are described, for example, in G. T. Hermanson, “Bioconjugate Techniques” (Academic Press, San Diego, 1996), ch. 8, pp. 373-400; ch. 113, pp. 570-575. A number of reactions, employing various functional groups, can be employed for linking compounds to biotin.

Any receptor-bearing cell can be targeted. The cell to be targeted can be, but is not limited to, a liver cell, a malignant cell, a cell that is a component of the central nervous system, or a cell that is an endothelial cell of the blood-brain barrier.

For direct delivery of drugs into cells such as cancer cells that express or overexpress the transferrin receptor (TfR) and/or the insulin receptor (IR), after the specific antibody-receptor interaction occurs, the whole complex including the carried agent will be internalized by receptor-mediated endocytosis which represents a highly efficient internalization pathway In this case the compound will remain inside the cells and will exert its function there.

The brain delivery characteristics of an antibody construct according to the present invention have been determined with its initial application in delivery to the brain of an anti-HIV peptide nucleic acid, an 18-mer antisense to the rev gene of HIV-1 with the structure 5′-biotin-CTCCGCTTCTTCCTGCCA-Tyr-Lys-CONH₂-3′.

VI. Screening Methods

Another aspect of the present invention is a method for screening a compound for cytotoxicity. In general, this method comprises the steps of:
(1) linking the compound to biotin or to a biotin analogue to form a conjugate recognized by avidin or streptavidin or their derivatives;
(2) binding the conjugate to a fusion protein or antibody construct according to the present invention as described above;
(3) binding the fusion protein or antibody construct bound to the conjugate to the surface of a cell in which cytotoxicity is to be screened;
(4) allowing the conjugate bound to the fusion protein or antibody construct to undergo antibody-receptor-mediated endocytosis; and
(5) determining the cytotoxicity of the compound by determining the survival of cells penetrated by the compound with the survival of a control sample of cells to which the fusion protein bound to the conjugate has not been targeted to determine the cytotoxic effect of the compound upon endocytosis.

The cell used for screening can be any receptor bearing cell. For example, the cell can be a liver cell, a malignant cell, or a cell that is a component of the central nervous system.

The compound for which cytotoxicity is to be screened can be as described above under “Targeting Methods.”

The invention is illustrated by the following Examples. These Examples are presented for illustration only and is not intended to limit the invention.
(1) linking the compound to biotin or to a biotin analogue to form a conjugate recognized by avidin or streptavidin or their derivatives;
(2) binding the conjugate to a fusion protein or antibody construct according to the present invention as described above;
(3) binding the fusion protein or antibody construct bound to the conjugate to the surface of a cell in which cytotoxicity is to be screened;
(4) allowing the conjugate bound to the fusion protein or antibody construct to undergo antibody-receptor-mediated endocytosis; and
(5) determining the cytotoxicity of the compound by determining the survival of cells penetrated by the compound with the survival of a control sample of cells to which the fusion protein bound to the conjugate has not been targeted to determine the cytotoxic effect of the compound upon endocytosis.

The cell used for screening can be any receptor bearing cell. For example, the cell can be a liver cell, a malignant cell, or a cell that is a component of the central nervous system.

The compound for which cytotoxicity is to be screened can be as described above under “Targeting Methods.”

The invention is illustrated by the following Examples. These Examples are presented for illustration only and is not intended to limit the invention.

### EXAMPLE 1

### Construction, Expression and Characterization of a Transferrin Receptor-Specific Antibody Fusion Protein Containing Chicken Avidin

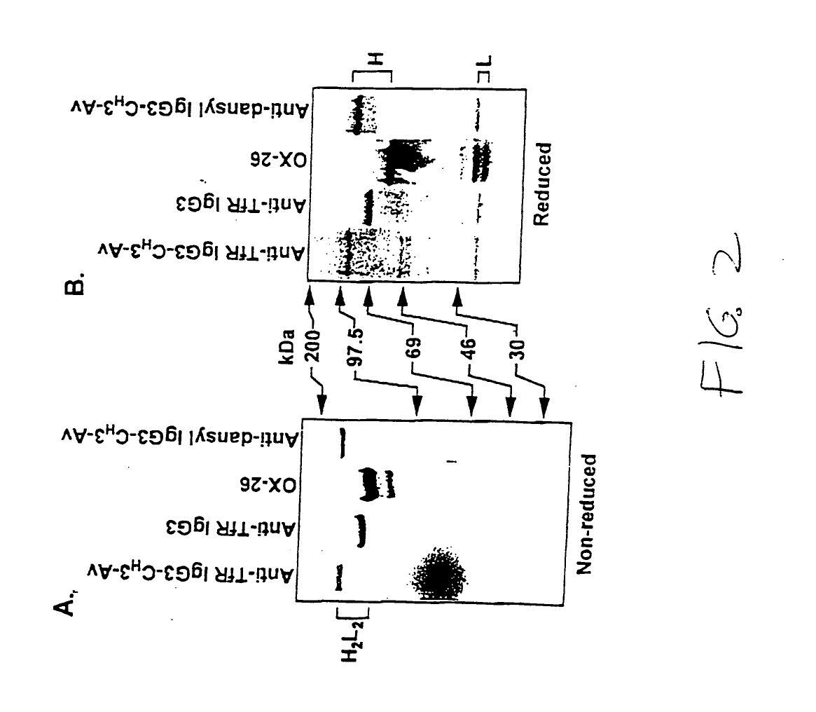

Construction, expression, and in vitro properties of mouse/human anti-TfR IgG3-C_{H}3-Av. The strategy for the expression of a transferrin receptor (TfR)-specific antibody fusion protein containing chicken avidin (Av), referred to as “anti-TfR IgG3-C_{H}3-Av” is illustrated in FIG. 1. Clones expressing anti-TfR IgG3-C_{H}3-Av fusion proteins were identified by an Enzyme-linked Immunosorbent Assay (ELISA) and biosynthetically labeled by growth in the presence of ³⁵S-methionine. SDS-PAGE analysis of the secreted ³⁵S-methionine labeled proteins under non-reducing conditions (FIG. 2A), showed the anti-TfR IgG3-C_{H}3-Av to have a molecular weight of approximately 200 kDa, the size expected for a complete antibody with 2 molecules of Av attached. This corresponds to the antibody construct described above and is a molecule with two heavy (H) chains and two light (L) chains. Following reduction, H and L chains of the expected molecular weight were observed (FIG. 2B). Anti-TfR IgG3-C_{H}3-Av purified from culture supernatants using affinity chromatography was also shown to be approximately 200 kDa (data not shown).

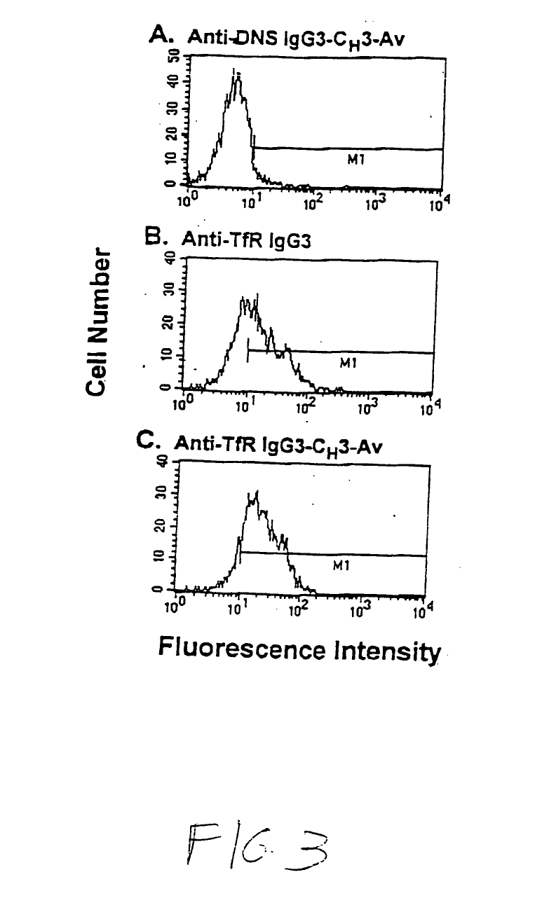

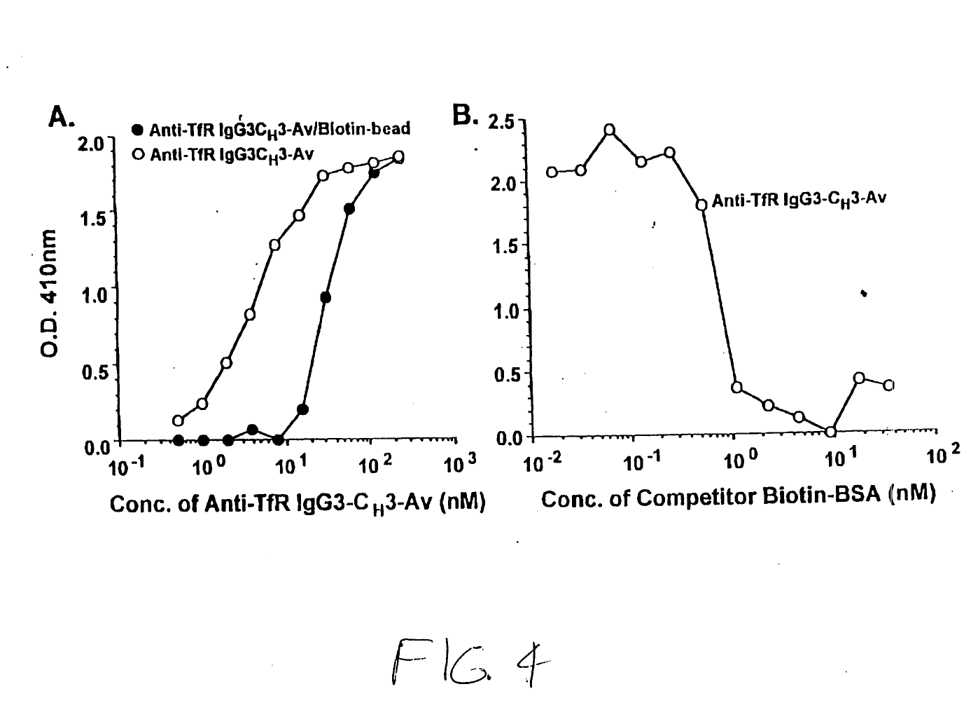

Flow cytometry using the rat myeloma cell line Y3-Ag1.2.3 showed that anti-TfP IgG3-C_{H}3-Av bound to the TfR expressed on the cell surface to the same extent as the anti-TfR Ab with the same variable region but lacking Av (FIG. 3). An irrelevant Ab (anti-hapten) fused to Av fail to bind. Anti-TfR IgG3-C_{H}3-Av also bound to biotinylated BSA coated on the surface of a microtiter plate in a dose-dependent manner (FIG. 4A). This binding activity could be removed by preincubation with biotin acrylic beads. In addition, soluble biotin-BSA inhibited the binding of anti-TfR IgG3-C_{H}3-Av to coated plates with 50% inhibition seen at an inhibitor concentration of 0.4 mM (FIG. 4B).

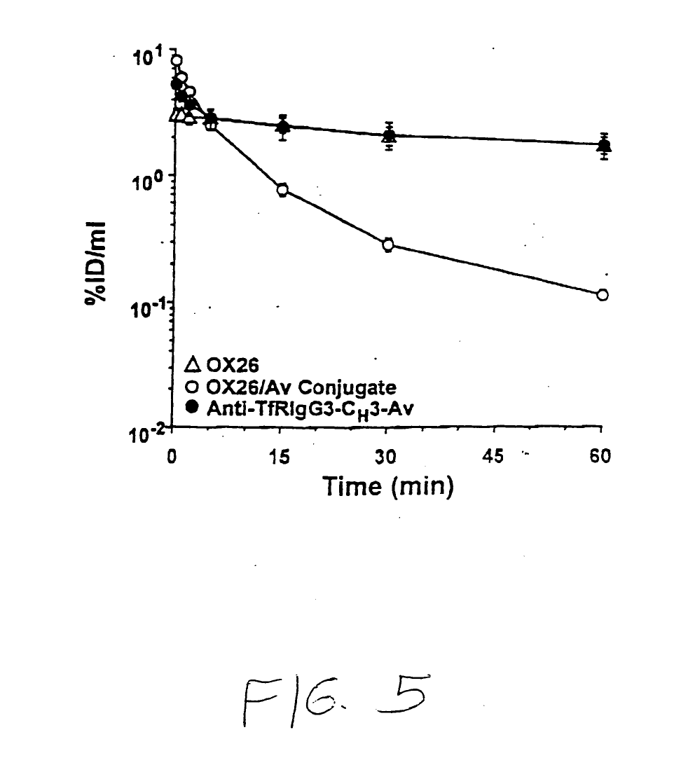

Pharmacokinetics and brain delivery of [³H]biotin bound to anti-TfR IgG3-C_{H}3-Av. Rats were injected intravenously with OX-26 (IgG2a anti-TfR) (46) labeled by iodination, or with OX-26 chemically conjugated to Av or anti-TfR IgG3-C_{H}3-Av labeled by incubation with [³H]-biotin and the radioactivity followed for 60 min (FIG. 5). [³H]-biotin bound to the OX-26/Av chemical conjugate was removed rapidly from the plasma compartment, while the rate of removal of [³H]-biotin bound to anti-TfR IgG3-C_{H}3-Av is similar to that of [¹²⁵I] labeled OX-26 (FIG. 5). The corresponding pharmacokinetic parameters obtained by fitting the data to a biexponential equation are given in Table 1. These data show that [³H]-biotin bound to anti-TfR IgG3-C_{H}3-Av is cleared from the peripheral compartment 5.8 fold more slowly than [³H]-biotin bound to the OX-26/Av chemical conjugate. The plasma “area under the curve” (AUC) of [³H]-biotin bound to the anti-TfR IgG3-C_{H}3-Av for the period of 0 to 60 min was increased by a factor of 2.8 compared to that of [³H]-biotin bound to the OX-26/Av conjugate, as a consequence of both a longer half-life of elimination (80.6±4.8 min vs. 20.5±2.2 min) and an increased “mean residence time” (MRT) (114±7 min vs. 16.0±1.3 min). Brain uptake of [³H]-biotin bound to anti-TfR IgG3-C_{H}3-Av was increased by a factor of 6.1 compared to that of the OX-26/Av conjugate (Table 1) reflecting both a 2.6-fold increase in the BBB permeability surface (PS) product (2.25±0.65 μl min⁻¹ g⁻¹ vs. 0.85±0.02 μl min⁻¹ g⁻¹) and the higher AUC. Systemic clearance of [³H]-biotin bound to anti-TfR IgG3-C_{H}3-Av is by the liver, which cleared 5.6±0.7% ID/g within 60 min following an intravenous injection.

Stability of [³H]-biotin-anti-TfR IgG3-C_{H}3-Av complex in serum. The serum stability of the [³H]-biotin anti-TfR IgG3-C_{H}3-Av fusion protein complex was examined by fast protein liquid chromatography (FPLC) (data not shown). Examination of the FPLC profile indicated that more than 90% of the plasma radioactivity ([³H]-biotin) eluted as a high molecular weight complex 60 min after injection with little free [³H]-biotin detected in the serum.

Discussion

Following intravenous injection, biotin bound to Av is rapidly removed from plasma with a half life of 1.3 min (47). This rapid rate of plasma clearance has been attributed to the attached carbohydrate and the cationic charge of Av which has 9 lysine and 8 arginine residues leading to an isoelectric point (pI) of 10. It is not surprising that chemical conjugation of Av to OX-26 leads to a reduced plasma AUC and a marked reduction of brain targeting compared with OX-26 (Table 1) (25). It was therefore unexpected that genetic fulsion of Av to the human IgG3 would result in a protein with a half-life similar to that of unconjugated OX-26. In related studies we have shown that the half life of anti-TfR IgG3-C_{H}3-Av is similar to anti-TfR IgG3. It is difficult to explain why the antibody chemically conjugated to Av has such different pharmacokinetic properties compared to the antibody genetically fused to Av. Perhaps the chemical treatment per se partially denatures the conjugate leading to its more rapid clearance. Alternatively, the site of Av addition may make important contributions to the pharmacokinetic properties. The fusion proteins are homogeneous with one Av attached at the end of the heavy chain. The conjugated proteins would be expected to be heterogeneous, varying both in the site and number of attached Av.

The amount of a drug delivered to the brain is typically expressed as the % ID/g which is a function of the BBB penetration (PS) and its persistence in the plasma (AUC) (25). The more efficient brain uptake of anti-TfR IgG3-C_{H}3-Av with an accumulation of 0.25% ID/g at 60 min after the intravenous bolus reflects both its improved PS and AUC compared to the chemical conjugate. This brain concentration is 3 fold higher than the brain uptake after 60 min of the classical neuroactive alkaloid morphine (0.081% ID/g) (48) and is comparable to that of OX-26.

Anti-TfR IgG3-C_{H}3-Av should serve as a universal vector for targeting the brain with a vast array of different compounds including chemicals, proteins and DNA. Although we have focused our discussion on brain targeting, we would like to stress that anti-TfR IgG3-C_{H}3-Av can be useful not only to target the brain but also other structures of the central nervous system (CNS) such as the cerebellum and spinal cord which are also limited by the BBB. Therefore, the results presented here suggest that our novel universal vector will have a large number of potential applications in the diagnosis and/or therapy of various CNS disorders.

Experimental Protocol

Vector construction. The anti-TfR IgG3-C_{H}3-Av heavy chain vector was constructed by the substitution of the variable region of anti-dansyl (5-dimethylamino naphthalene 1-sulfonyl chloride) IgG3-C_{H}3-Av fusion heavy chain (47) with the variable region of the heavy chain of anti-rat TfR mAb OX-26 (46) (FIG. 1).

Transfection and initial characterization of anti-rat TfR IgG3-C_{H}3-Av. All cells were cultured in Dulbecco's Modified Eagle Medium (DMEM; GIBCO BRL, Grand Island, N.Y.) with 5% calf serumn (HyClone, Logan, Utah). A cell line that produces high levels of the anti-TfR kappa light chain, TAUD3.1, was obtained by transfecting P3X63Ag8.653 by electroporation with a chimeric mouse/human k light chain gene with the variable region of OX-26 (FIG. 1), selecting with 0.33×HXM (30×HXM contains 3.3 mM hypoxanthine, 49.3 mM xanthine, 0.52 mM mycophenolic acid, and 0.1N NaOH) and detecting stable transfectants secreting L chain by ELISA (49). One light chain expressing transfectant, TAUD3.1, was electroporated with the anti-rat TfR IgG3-C_{H}3-Av heavy chain gene (49), stable transfectants were selected with 5 mM histidinol (Sigma Chemical, St. Louis, Mo.) and screened by an ELISA for the secretion of heavy chain (49). The fusion protein biosynthetically labeled with ³⁵S-Methionine (ICN, Irvine, Calif.) was immunoprecipitated using rabbit anti-human IgG and a 10% suspension of staphylococcal protein A (IgGSorb, The Enzyme Center, Malden, Mass.) and then analyzed by SDS-PAGE with/without 2-mercaptoethanol. The fusion protein was purified from culture supernatants using protein G immobilized on Sepharose 4B fast flow (Sigma Chemical). Purity was assessed by Coomassie blue staining of SDS-PAGE gels. Protein concentrations were determined by bicinchoninic acid based protein assay (BCA Protein Assay. Pierce Chemical Co., Rockford, Ill.) and ELISA.

Antigen binding study. The binding of anti-TfR IgG3-C_{H}3-Av to the TfR was studied by flow cytometry using the rat myeloma cell line Y3-Ag1.2.3. Cells (1×10⁶) were incubated with 1 μg of anti-TfR IgG3-C_{H}3-Av, anti-DNS IgG3-C_{H}3-Av (negative control), or anti-rat TfR IgG3 (positive control), in a volume of 100 μl for 2 h at 4° C., washed, incubated 2 h at 4° C. with FITC-labeled goat anti-human IgG (Pharmingen, San Diego, Calif.) and analyzed by flow cytometry (Becton-Dickinson, Mountain View, Calif.).

Biotinylated Human Serum Albumin Binding Assays. All steps were carried out in phosphate buffer saline (PBS) and plates were washed six times between each step with the same buffer. 96-well plates were coated with 50 μl/well biotinylated-BSA (Sigma Chemical) (biotin:BSA ratio=11:1, 5 μg/ml) overnight at 4° C. then blocked with 100 μl/well 3% BSA (overnight at 4° C.) (47). All fusion proteins (by duplicate) were diluted and applied in a volume of 50 μl/well and after overnight incubation at 4° C., goat anti-human κ-alkaine phosphatase conjugate (Sigma Chemical) was added followed by 50 μl of the substrate p-nitrophenyl phosphate at 0.5 mg/ml in diethanolamine buffer (pH 9.6) (Sigma Chemical). The optical density was read at 410 nm. To determine if anti-Tfe IgG3-C_{H}3-Av could be removed with biotin acrylic beads, varying concentrations of the fusion protein (0.5-250 nM) were pre-incubated with biotin acrylic beads (Sigma Chemical) (5 μl) at room temperature for 30 min. After brief centrifugation, the presence of the fusion protein in the supernatants was quantitated by ELISA as described above. For a competition ELISA, anti-rat TfR IgG3-C_{H}3-Av (2.5 nM) was preincubated with various concentrations of biotin-BSA (35.4 pM-36.3 nM) at 37° C. for 2 hours and then ELISA was performed as described.

Pharmacokinetics and brain delivery of [3H]-biotin bound to anti-TfR IgG3-C_{H}3Av. Male Sprague-Dawley rats (three rats per group) weighing 220 to 230 g purchased from Samyook Experimental Animals (Buann, Korea) were anesthetized with ketamine (100 mg/kg) and xylazine (2 mg/kg) by intramuscular injection. The left femoral vein was cannulated with PE50 tubing and injected with 0.2 ml of Ringer-N-[2-hydroxyethyl]piperazine-N′-[2-ethanesulfonic acid] (HEPES, pH 7.4) containing 0.1% native rat serum albumin and 5 μCi (0.1 nmol) of [³H]-biotin (Du Pont NEN Research Products, Bukyungsa, Korea) mixed with 20 μg of antibody-fusion proteins (0.1 nmol) or chemical conjugate (OX-26/Av). OX-26 was directly labeled with [¹²⁵I] (50). Blood samples (0.3 ml) were collected via a heparinized PE50 cannula implanted in the left femoral vein at 0.25, 1, 2, 5, 15, 30, and 60 min after the intravenous injection. After each blood sampling, the blood volume was replaced with the same volume of normal saline, and plasma was separated by centrifugation. The animals were decapitated after 60 min and the brain was removed and weighed. The plasma and brain samples were solubilized with Soluene-350 (Packard Instrument Co., Saehan, Korea) and neutralized with glacial acetic acid prior to liquid scintillation counting. The pharmnacokinetic parameters were calculated by fitting plasma radioactivity data to a bi-exponential equation, as described previously (25). The BBB permeability-surface area (PS) product of [³H]-biotin bound to anti-TfR IgG3-C_{H}3-Av was calculated as described (25) from the plasma concentrations, the apparent brain volume of distribution (V_{D}), and the plasma volume in brain (10 μl/g). The % injected dose (ID) delivered per gram brain was computed from the PS product and the 60 min area under the plasma concentration curve (AUC), as described previously (25).

Stability of [³H]-biotin fusion protein complex in serum. The serum stability of the [³H]-biotin anti-TfR IgG3-C_{H}3-Av complex was examined by fast protein liquid chromatography (FPLC) using a Superose 6HR 10/30 column (Pharmacia Biotech, Uppsala, Sweden). A 50 μl aliquot of either 60 min serum samples, or of an in vitro preparation containing 7.5 μCi of [³H]-biotin and 30 μl of anti-TfR IgG3-C_{H}3-Av as a control (injectate) was injected into the column. The samples were passed through the column in the presence of 0.01M PBS (pH 7.4) containing 0.05% Tween-20 at a flow rate 0.25 ml/min. Fractions (0.5 ml) were collected and the radioactivity of each fraction was counted on a Packard Liquid Scintillation Analyzer (Model A2100 TR).

### REFERENCES

The following references are referred in Example 1 and elsewhere in the application, except in Example 2, which has additional references identified by number.
1. Crone, C., and Olesen, S. P. 1982. Electrical resistance of brain microvascular endothelium. Brain Research 241: 49-55.
2. Butt, A. M., Jones, H. C., and Abbott, N. J. 1990. Electrical resistance across the blood-brain barrier in anaesthetized rats: a developmental study. J Physiol (Lond) 429: 47-62.
3. Pardridge, W. M. 1991. pp. 123-148, in Peptide Drug Delivery to the Brain, Raven Press, New York.
4. Brightman, M. W., and Tao-Cheng, J. H. 1993. Tight Junctions of Brain Endothelium and Epithelium. pp. 107-125, in The Blood-Brain Barrier: Cellular and Molecular Biology, Pardridge, W. M. (ed.) Raven Press, New York.
5. Abbott, N. J., and Romero, I. A. 1996. Transporting therapeutics across the blood-brain barrier. Mol Med Today 2:106-113.
6. Shapiro, W. R., and Shapiro, J. R. 1986. Principles of brain tumor chemotherapy. Semin Oncol 13: 56-69.
7. Olson, L., Backlund, E. O., Ebendal, T., Freedman, R., Hamberger, B., Hansson, P., Hoffer, B., Lindblom, U., Meyerson, B., Stromberg, I., et al. 1991. Intraputaminal infusion of nerve growth factor to support adrenal medullary autografts in Parkinson's disease. One-year follow-up of first clinical trial. Arch Neurol 48: 373-381.
8. Olson, L., Nordberg, A., Vonholst, H., Backman, L., Ebendal, T., Alafuzoff, I., Amberla, K., Hartvig, P., Herlitz, A., Lilja, A., Lundqvist, H., Langstrom, B., Meyerson, B., Persson, A., Viitanen, M., Winblad, B., and Seiger, A. 1992. Nerve growth factor affects C-11-nicotine binding, blood flow, eeg, and verbal episodic memory in an Alzheimer patient. J Neur TR-P 4: 79-95.
9. Gash, D. M., Zhang, Z., and Gerhardt, G. 1998. Neuroprotective and neurorestorative properties of GDNF. Ann Neurol 44: S121-125.
10. Rosenberg, M. B., Friedmann, T., Robertson, R. C., Tuszynski, M., Wolff, J. A., Breakefield, X. O., and Gage, F. H. 1988. Grafting genetically modified cells to the damaged brain: restorative effects of NGF expression. Science 242: 1575-1578.
11. Kordower, J. H., Fiandaca, M. S., Notter, M. F., Hansen, J. T., and Gash, D. M. 1990. NGF-like trophic support from peripheral nerve for grafted rhesus adrenal chromaffin cells. J Neurosurg 73: 418-428.
12. Duffy, K. R., and Pardridge, W. M. 1987. Blood-brain barrier transcytosis of insulin in developing rabbits. Brain Research 420: 32-38.
13. Fishman. J. B., Rubin, J. B., Handrahan, J. V., Connor, J. R., and Fine, R. E. 1987. Receptor-mediated transcytosis of transferrin across the blood-brain barrier. J Neurosci Res 18: 299-304.
14. Rosenfeld, R. G., Pham, H., Keller, B. T., Borchardt, R. T., and Pardridge, W. M. 1987. Demonstration and structural comparison of receptors for insulin-like growth factor-I and -II (IGF-I and -II) in brain and blood-brain barrier. Biochem Biophys Res Commun 149: 159-166.
15. Shin, S. U., Friden, P., Moran, M., Olson, T., Kang, Y. S., Pardridge, W. M., and Morrison, S. L. 1995. Transferrin-antibody fusion proteins are effective in brain targeting. Proc Natl Acad Sci USA 92: 2820-2824.
16. Shin, S. U., Friden, P., Moran, M., and Morrison, S. L. 1994. Functional properties of antibody insulin-like growth factor fusion proteins. J Biol Chem 269: 4979-4985.
17. Friden. P. M., Walus, L. R., Musso, G. F., Taylor, M. A., Malfroy, B., and Starzyk, R. M. 1991. Anti-tansferrin receptor antibody and antibody-drug conjugates cross the blood-brain barrier. Proc Natl Acad Sci USA 88: 4771-4775.
18. Friden. P. M., Walus, L. R., Watson, P., Doctrow, S. R., Kozarich, J. W., Bäckman, C., Bergman, H., Hoffer, B., Bloom, F., and Granholm, A. C. 1993. Blood-brain barrier penetration and in vivo activity of an NOF conjugate. Science 259: 373-377.
19. Walus. L. R., Pardridge, W. M., Starzyk, R. M., and Friden, P. M. 1996. Enhanced uptake of rsCD4 across the rodent and primate blood-brain barrier after conjugation to anti-transferrin receptor antibodies. J Pharmacol Exp Ther 277: 1067-1075.
20. Bullard, D. E., Bourdon. M., and Bigner, D. D. 1984. Comparison of various methods for delivering radiolabeled monoclonal antibody to normal rat brain. J Veurosurg 61: 901-911.
21. Kordower, J. H., Charles, V., Bayer, R., Bartus, R. T., Putney, S., Walus, L. R., and Friden, P. M. 1994. Intravenous administration of a transferrin receptor antibody-nerve growth factor conjugate prevents the degeneration of cholinergic striatal neurons in a model of Huntington disease. Proc Natl Acad Sci USA 91: 9077-9080.
22. Granholm, A. C., Albeck, D., Backman, C., Curtis, M., Ebendal, T., Friden, P., Henry, M., Hoffer, B., Kordower, J., Rose, G. M., Saderstrom, S., and Bartus, R. T. 1998. A non-invasive system for delivering neural growth factors across the blood-brain barrier: a review. Rev Neurosci 9: 31-55.
23. McGrath, J. P., Cao, X., Schutz, A., Lynch, P., Ebendal, T., Coloma. M. J., Morrison, S. L., and Putney, S. D. 1997. Bifunctional fusion between nerve growth factor and a transferrin receptor antibody. J Neurosci Res 47: 123-133.
24. Yoshikawa, T., and Pardridge, W. M. 1992. Biotin delivery to brain with a covalent conjugate of avidin and a monoclonal antibody to the transferrin receptor. J Pharmacol Exp Ther 263: 897-903.
25. Kang, Y. S., Bickel, U., and Pardridge, W. M. 1994. Pharmacokinetics and saturable blood-brain barrier transport of biotin bound to a conjugate of avidin and a monoclonal antibody to the transferrin receptor. Drug Metabolism and Disposition 22: 99-105.
26. Fan, Z., Baselga, J., Masui, H. and Mendelsohn, J. 1993. Antitumor effect of anti-epidermal growth factor receptor monoclonal antibodies plus cis-diamminedichloroplatinum on well established A431 cell xenografts. Cancer Res., 53: 4637-4642
27. Shimizu, N. Chen, J., Gamou, S., and Takayanagi, A. 1996. Immunogene approach toward cancer therapy using erythrocyte growth factor receptor-mediated gene delivery. Cancer Gene Ther., 3: 113-120.
28. Cristiano, R. J. and Roth, J. A. 1996. Epidermal growth factor mediated DNA delivery into lung cancer cells via the epidermal growth factor receptor. Cancer Gene Ther., 3: 4-10.
29. Curiel, D. T., Agarwal, S., Wagner, E., and Cotten, M. 1991. Adenovirus enhancement of transferrin-polylysine-mediated gene delivery. Proc. Natl. Acad. Sci. USA., 88: 8850-8854.
30. Wagner, E., Cotten, M., Foisner, R., and Bimnstiel, M. L. 1991. Transferrin-polycation-DNA complexes: the effect of polycations on the structure of the complex and DNA delivery to cells. Proc. Natl. Acad. Sci. USA., 88: 4255-4259.
31. Watanabe, N., Sato, Y., Yamauchi, N., and Niitsu, Y. 1998. Gene delivery into human cancer cells via transferrin receptor. Japanese J. Clin. Med., 56: 724-730.
32. Smith, J. S. Keller, J. R., Lohrey, N. C., McCauslin, C. S., Ortiz, M., Cowan, K., and Spence, S. E. 1999. Redirected infection of directly biotinylated recombinant adenovirus vectors through cell surface receptors and antigens. Proc. Natl. Acad. Sci. USA., 96: 8855-8860.
33. Sweeney, E. B. and Murphy, J. R. 1995. Diphtheria toxin-based receptor-specific chimaeric toxins as targeted therapies. Essays Biochem., 30: 119-131.
34. Rybak, S. M., Saxena, S. K, Ackerman, E. J., and Youle, R. J. 1991. Cytotoxic potential of ribonuclease and ribonuclease hybrid proteins. J. Biol. Chem., 266: 21202-21207.
35. Psarras, K., Ueda. M., Yamamura. T., Ozawa, S., Kitajima, M., Aiso, S., Komatsu. S. and Seno, M. 1998. Human pancreatic RNase 1-human epidermal growth factor fusion: an entirely human ‘immunotoxin analog’ with cytotoxic properties against squamous cell carcinomas. Protein Eng., 11: 1285-1292.
36. Nichols, J., Foss, F., Kuzel, T. M., LeMaistre, C. F., Platanias, L., Ratain, M. J., Rook, A., Saleh, M., and Schwartz, G. 1997. Interleukin-2 fusion protein: an investigational therapy for interleukin-2 receptor expressing malignancies. Eur. J. Cancer., 33 Suppl 1: S34-36.
37. LeMaistre, C. F., Saleh, M. N., Kuzel, T. M., Foss, F., Platanias, L. C., Schwartz, G., Ratain M., Rook, A., Freytes, C. O., Craig, F., Reuben, J., and Nichols, J. C. 1998. Phase I trial of a ligand fusion-protein (DAB389IL-2) in lymphomas expressing the receptor for interleukin-2. Blood, 91: 399-405.
38. Dowlati, A., Loo, M., Bury, T., Fillet, G., and Beguin, Y. 1997. Soluble and cell-associated transferrin receptor in lung cancer. Br. J. Cancer., 75: 1802-1806.
39. May, W. S., Jr. and Cuatrecasas, P. 1985. Transferrin receptor: its biological significance. J. Membr. Biol., 88: 205-215.
40. Habelshaw, H. A., Lister, T. A., and Stansfeld, A. G. 1983. Correlation of transferrin receptor expression with histological class and outcome in non-Hodgkin lymphoma. Lancet., 1: 498-500.
41. Beguin, Y., Lampertz, S., Degroote, D., Igot, D., Malaise, M., and Fillet, G. 1993. Soluble Cd23 and Other Receptors (Cd4, Cd8, Cd25, Cd71) In Serum Of Patients With Chronic Lymphocytic Leukemia. Leukemia., 7: 2019-2025.
42. Gatter, K. C. Brown, G. Trowbridge, I. S., Woolston, R. E., and Mason, D. Y. 1983. Transferrin receptors in human tissues: their distribution and possible clinical relevance. J. Clin. Pathol., 36: 539-545.
43. Lloyd, J. M. O'Dowd, T., Driver, M., and Tee, D. E. 1984. Demonstration of an epitope of the transferrin receptor in human cervical epithelium—a potentially useful cell marker. J. Clin. Pathol., 37: 131-135.
44. Raaf. H. N., Jacobsen. D. W., Savon, S., and Green, R. 1993. Serum Transferrin Receptor Level Is Not Atered In Invasive Adenocarcinoma Of the Breast. Amer. J Clin Pathol., 99: 232-237.
45. Tanoguchi, H., Tachibana, M., and Murai, M. 1997. Autocrine growth induced by transferrin-like substance in bladder carcinoma cells. Br. J. Cancer., 76: 1262-1270.
46. NormandSdiqui, N., and Akhtar, S. 1998. Oligonucleotide delivery: Uptake of rat transferrin receptor antibody (OX-26) conjugates into an in vitro immortalised cell line model of the blood-brain barrier. Int J Pharm 163: 63-71.
47. Shin. S. U., Wu, D., Ramanathan, R., Pardridge, W. M., and Morrison, S. L. 1997. Functional and pharmacokinetic properties of antibody-avidin fusion proteins. J Immunol 158: 4797-4804.
48. Wu, D., Kang, Y. S., Bickel, U. and Pardridge, W. M. 1997. Blood-brain barrier permeability to morphine-6-glucuronide is markedly reduced compared with morphine. Drug Metab Dispos 25: 768-771.
49. Shin, S. U., and Morrison, S. L. 1989. Production and properties of chimeric antibody molecules. Methods Enzymol 178: 459-476.
50. Pardridge, W. M., Buciak, J. L., and Friden, P. M. 1991. Selective transport of an anti-transferrin receptor antibody through the blood-brain barrier in vivo. J Pharmacol Exp Ther 259: 66-70.
   Figure Legends.

The following are figure legends for the figures of Example 1.

FIG. 1. Schematic diagram of the construction and expression of the anti-TfR IgG3-C_{H}3-Av fusion protein. Using convenient restriction sites, the anti-Tfe IgG3-C_{H}3-Av heavy chain expression vector was constructed by substituting the variable region of the anti-dansyl IgG3-C_{H}3-Av heavy chain with that of an antibody specific for the rat TfR (OX-26). TAUD3.1, a transfectant of P3X63Ag8.653 expressing a light chain with the OX-26 variable region was used as a recipient for transfection of the anti-TfR IgG3-C_{H}3-Av heavy chain expression vector.

FIG. 2. SDS-PAGE analysis of the anti-TfR IgG3-C_{H}3-Av fusion protein. Secreted anti-TfR IgG3-C_{H}3-Av biosynthetically labeled with ³⁵S-methionine was immunoprecipitated using anti-human IgG and staphylococcal protein A and analyzed by SDS-PAGE under non-reducing (A) and reducing (B) conditions. Included for comparison are anti-TfR IgG3 without attached Av, OX-26 (the murine IgG2a anti-TfR which donated the variable regions), and a previously characterized anti-dansyl IgG3-C_{H}3-Av. The positions of the MW standards are indicated at the side.

FIG. 3. Flow cytometry demonstrating the specificity of the anti-rat TfR IgG3-C_{H}3-Av for the TfR expressed on the surface of rat Y3-Ag1.2.3 cells. The cells were incubated with either negative control anti-DNS IgG3-C_{H}3-Av (A), positive control anti-rat TfR IgG3 (B), or the anti-rat TfR IgG3-C_{H}3-Av fusion protein (C), followed by FITC-labeled goat anti-human IgG.

FIG. 4. The binding of anti-TfR IgG3-C_{H}3-Av to biotinylated BSA coated microtiter plates. (A) Anti-TfR IgG3-C_{H}3-Av was added at varying concentrations with/without previous incubation with biotin acrylic beads and the bound protein detected using anti-kappa conjugated with alkaline phosphatase. (B) Anti-TfR IgG3-C_{H}3-Av (2.5 nM) preincubated with varying concentrations of biotinylated BSA was added to the biotinylated BSA coated microtiter plates and bound Ab detected using anti-kappa conjugated with alkaline phosphatase.

FIG. 5. Plasma clearance of proteins: The plasma profiles of ¹²⁵I-OX-26 and of [³H]-biotin bound to either the OX-26/Av conjugate, or anti-TfR IgG3-C_{H}3-Av fusion protein were analyzed. The open triangles represent ¹²⁵I-OX-26, the open circles anti-TfR OX-26/Av conjugate, the filled circles anti-TfR IgG3-C_{H}3-Av. % ID/ml represents percentage of injected dose per ml plasma.

TABLE 1
PHARMACOKINETIC PARAMETERS^{a} FOR [¹²⁵I] OX-26 AND [³H] BIOTIN
BOUND TO THE OX-26/Av CONJUGATE OR ANTI-TfR IgG3-CH³-Av
60 MINUTES AFTER INTRAVENOUS INJECTION IN THE RAT

| | | [³H] Biotin Carrier | |
|---|---|---|---|
| Parameter^{b} | [¹²⁵I] OX-26 | OX-26 Av Conjugate | Anti-TfR IgG3-C_{H}³-Av |
| A₁ (% ID/ml) | 2.99 ± 0.38 | 6.75 ± 0.43 | 2.91 ± 0.32 |
| A₂ (% ID/ml) | — | 0.62 ± 0.20 | 2.75 ± 0.58 |
| K₁ (min⁻¹) | 0.01 ± 0.001 | 0.25 ± 0.02 | 0.58 ± 0.07 |
| K₂ (min⁻¹) | — | 0.035 ± 0.003 | 0.009 ± 0.001 |
| t_{1/2}¹ (min): Distribution | 65 ± 5 | 2.82 ± 0.22 | 1.24 ± 0.15 |
| t_{1/2}² (min): Elimination | 81 ± 5 | 20.5 ± 2.2 | 80.6 ± 4.8 |
| AVCₒ₋₆₀ (% IDmin/ml) | 132 ± 19 | 48.5 ± 4.0 | 134 ± 29 |
| AVC_{o-∞} (% IDmin/ml) | 282 ± 52 | 50.4 ± 5.0 | 332 ± 89 |
| Vss (ml/kg) | 133 ± 15 | 143 ± 17 | 172 ± 25 |
| CLss (ml/min/kg) | 1.45 ± 0.23 | 8.94 ± 0.61 | 1.54 ± 0.29 |
| MRT (min) | 93 ± 6 | 16.0 ± 1.3 | 114 ± 7 |
| Brain Vo (μg/min/g) | 169 ± 3 | 401 ± 48 | 91 ± 8 |
| PS (μl/g) | 1.92 ± 0.06 | 0.85 ± 0.02 | 2.25 ± 0.65 |
| Brain Uptake (% ID/g) | 0.27 ± 0.04 | 0.041 ± 0.004 | 0.25 ± 0.09 |
| ^{a}For the pharmacokinetic parameters the subscript 1 represents the distribution phase and the subscript 2 the elimination phase. A indicates the intercept value on the Y-axis in FIG. 5, K the transfer rate and CL the plasma clearance rate. AVCₒ₋₆₀ and AVC_{o-∞} are the first 60 minutes and steady-state area under the plasma concentration curve respectively. | | | |
| # Vss is the systemic volume of distribution, MRT the mean residence time, and Vo the brain volume of distribution. | | | |

^{b}Calculated from the data in FIG. 5 for a 60-min. period; therefore, the t_{1/2}² is considered as an estimate.

TABLE 2
ORGAN CLEARANCE AND DELIVERY OF [³H] BIOTIN
BOUND TO THE ANTI-IgG3-C_{H}3-Av FUSION PROTEIN

| | Organ Clearance | Uptake |
|---|---|---|
| Organ | (μl/min/g) | (% ID/g) |
| Brain | 2.25 ± 0.65 | 0.25 ± 0.09 |
| Lung | 2.54 ± 0.78 | 0.30 ± 0.06 |
| Heart | 1.18 ± 0.49 | 0.14 ± 0.05 |
| Kidney | 2.44 ± 0.69 | 0.37 ± 0.18 |
| Liver | 46.4 ± 12.8 | 5.60 ± 0.69 |

Measurements were made 60 min. after intravenous injection. Data are mean ± SE. (n = 3, rats)

TABLE 3
BRAIN UPTAKES OF BIOTIN-PNA WITH OR
WITHOUT ANTI-TfR IgG3-C_{H}3-Av

| | PS Product | Brain Uptake |
|---|---|---|
| Injectate | (μl/ml/g brain) | (% ID/g brain) |
| [¹²⁵I]-Biotin-PNA | 0.12 ± 0.01 | 0.0083 ± 0.0009 |
| Anti-TfR IgG3-C_{H}3- | 0.67 ± 0.09 | 0.12 ± 0.03 |
| Av/[¹²⁵I]-Biotin-PNA | | |

### EXAMPLE 2

### Use of Antibody Construct of Example 1 to Deliver Antisense Peptide Nucleic Acid to Brain

The antibody construct of Example 1 was used to deliver an 18-mer peptide nucleic acid with biotin at its 5′-end and lysine and tyrosine at its 3′-end to brain as a model for the treatment of HIV in brain. This peptide nucleic acid is an antisense peptide nucleic acid for the rev gene of HIV-1.

Efficient and specific targeting of an active agent to the desired site is a critical factor for the successful diagnosis and/or therapy of many diseases. One region of the body particularly difficult to target is the brain due to the presence of the high resistance blood-brain barrier (BBB) formed by tightly joined capillary endothelial cell membranes (1-5). The BBB effectively restricts transport from the blood of certain molecules, especially those that are water soluble and larger than several hundred daltons (6). In fact the clinical utility of many proteins of therapeutic interest for the brain is limited by their inability to cross the BBB. In some cases neurotrophic factors have been administered to the brain by invasive neurosurgical procedures or grafting neurotrophin-producing cells into brain sites (7-9).

The BBB has been shown to have specific receptors which allow the transport from the blood to the brain of several macromolecules including insulin (10), transferrin (Tf) with iron attached (11), and insulin-like growth factors (IGFs) (12). Therefore, one noninvasive approach for the delivery of drugs to the brain is to attach the agent of interest to a molecule with receptors on the BBB which would then serve as a vehicle for transport of the agent across the BBB (3, 13, 14). An alternative approach is the delivery of agents attached to an antibody specific for one of the BBB receptors. Indeed, both NGF and CD4 will cross the BBB when chemically conjugated to an antibody directed against the transferrin receptor (TfR) (15-17).

Despite the fact that antibodies normally are excluded from the brain (18), they can be an effective vehicle for the delivery of molecules into the brain parenchyma if they have specificity for receptors on the BBB. In fact, the intravenous injection of an anti-rat TfR antibody-NGF chemical conjugate prevented the loss of striatal choline acetyltransferase-immunoreactive neurons in a rat model of Huntington's disease and reversed the age-related cognitive dysfunction (19, 20). Recently a fusion protein with NGF attached to the N-terminus of an antibody directed against human TfR using genetic engineering techniques (21) showed both antigen binding and NGF activity suggesting its therapeutic utility. Although promising this approach requires that unique chimeric molecules be constructed for each specific application, is cumbersome and sometimes can lead to the decrease or loss of activity of one or both of the covalently conjugated partners. To overcome these limitations it is therefore desirable to develop a universal delivery system that eliminates the need to make a specific construct for each individual application.

The ideal brain delivery vehicle should be able to deliver many different compounds which are bound to the vehicle by high affinity noncovalent interactions such as those seen by avidin (Av) and biotin. Indeed, antibody-Av chemical conjugates have been used to deliver a mono-biotinylated drug (22). However, an important drawback of the chemical coupling procedure is the difficulty in producing a reproducible and homogeneous product. Genetic engineering provides an alternative approach for the large scale production homogeneous antibody-Av fusion proteins. The work of this Example describes the brain delivery characteristics of a TfR specific antibody containing chicken Av and its initial application in delivery to the brain of anti-HIV-1 peptide nucleic acid, an 18-mer antisense to the rev gene of HIV-1 with the structure 5′-biotin-CTCCGCTTCTTCCTGCCA-Tyr-Lys-CONH₂-3′ (biotin-PNA) (23). The fusion protein demonstrated superior [³H]-biotin uptake into brain parenchyma in comparison with the chemical conjugate. In addition, the brain uptake of anti-HIV-PNA was increased at least 15-fold when it was bound to the anti-rat TfR IgG3-C_{H}3-Av (“antibody construct”). As the brain is a shelter for HIV, the successful brain delivery of anti-HIV PNA with the antibody construct may provide an effective treatment for cerebral acquired immune deficiency syndrome (AIDS).

Materials and Methods

Vector construction, transfection, and initial characterization of anti-rat TfR IgG3-C_{H}3-Av were performed as in Example 1. The antigen binding study was performed as in Example 1. The biotinylated human serum albumin binding assays were done as in Example 1.

Pharmacokinetics and brain delivery of [³H]-biotin bound to anti-TfR IgG3-C_{H}3-Av. Male Sprague-Dawley rats (three rats per group) weighing 220 to 230 g purchased from Samyook Experimental Animals (Buann, Korea) were anesthetized with ketamine (100 mg/kg) and xylazine (2 mg/kg) by intramuscular injection. The left femoral vein was cannulated with PE50 tubing and injected with 0.2 ml of Ringer-N-[2-hydroxyethyl]piperazine-N′-[2-ethanesulfonic acid] (HEPES, pH 7.4) containing 0.1% native rat serum albumin and 5 μCi (0.1 nmol) of [³H]-biotin (Du Pont NEN Research Products, Bukyungsa, Korea) mixed with 20 μg of antibody-fusion proteins (0.1 nmol) or chemical conjugate (OX-26/Av). 5 μCi of [¹²⁵I]-biotin-PNA mixed with 20 μg of anti-TfR IgG3-C_{H}3-Av or 20 μg of [¹²⁵I]-anti-TfR IgG3-C_{H}3-Av. PNA, an 18-mer antisense to the rev gene of human immunodeficiency virus type 1, was custom synthesized by Millipore (Millipore Corporation, Bedford, Mass.) such that the 5′-end was biotinylated, and tyrosine and lysine were placed at the amidated 3′-end 5′-biotin-CTCCGCTTCTTCCTGCCA-Tyr-Lys-CONH₂ (23). OX-26 was labeled with [³H} succinimidyl propionate (Amersham Corp.) as described previously (27) and PNA was directly labeled with [¹²⁵I] (as described previously (23). Blood samples (0.3 ml) were collected via a heparinized PE50 cannula implanted in the left femoral vein at 0.25, 1, 2, 5, 15, 30, and 60 min after the intravenous injection. After each blood sampling, the blood volume was replaced with the same volume of normal saline, and plasma was separated by centrifugation. The animals were decapitated after 60 min and the brain was removed and weighed. The plasma and brain samples were solubilized with Soluene-350 (Packard Instrument Co., Saehan, Korea) and neutralized with glacial acetic acid prior to liquid scintillation counting. The other peripheral tissues such as liver, kidney, lung, and heart were also removed and weighted, and their radioactivities were counted. The pharmacokinetic parameters were calculated by fitting plasma radioactivity data to a mono- or bi-exponential equation, as described previously (22). The BBB permeability-surface area (PS) product of [³H]-biotin or [¹²⁵I]-biotin-PNA bound to anti-TfR IgG3-C_{H}3-Av was calculated as described (22) from the plasma concentrations, the apparent brain volume of distribution (V_{D}), and the plasma volume in brain (10 μl/g). The % injected dose (ID) delivered per gram brain was computed from the PS product and the 60 min area under the plasma concentration curve (AUC), as described previously (28).

Stability of the [³H]-biotin fusion protein in complex was monitored as in Example 1.

Results

Construction, Expression, and in Vitro Properties of Mouse/Human Anti-TfR IgG3-C_{H}3-Av.

The strategy for the expression of a transferrin receptor (TfR)-specific antibody fusion protein containing chicken avidin (Av), referred to as “anti-TfR IgG3-C_{H}3-Av” is illustrated in FIG. 1. Clones expressing anti-TfR IgG3-C_{H}3-Av fusion proteins were identified by an Enzyme-linked Immunosorbent Assay (ELISA) and biosynthetically labeled by growth in the presence of ³⁵S-methionine. SDS-PAGE analysis of the secreted ³⁵S-methionine labeled proteins under non-reducing conditions (FIG. 2A), showed the anti-TfR IgG3-C_{H}3-Av to have a molecular weight of approximately 200 kDa, the size expected for a complete antibody with 2 molecules of Av attached. This corresponds to the antibody construct described above and is a molecule with two heavy (H) chains and two light (L) chains. Following reduction, H and L chains of the expected molecular weight were observed (FIG. 2B). Anti-TfP IgG3-C_{H}3-Av purified from culture supernatants using affinity chromatography was also shown to be approximately 200 kDa (data not shown).

Flow cytometry using the rat myeloma cell line Y3-Ag1.2.3 showed that anti-TfR IgG3-C_{H}3-Av bound to the TfR expressed on the cell surface to the same extent as the anti-TfR Ab with the same variable region but lacking Av (FIG. 3). An irrelevant Ab (anti-hapten) fused to Av fail to bind. Anti-TfR IgG3-C_{H}3-Av also bound to biotinylated BSA coated on the surface of a microtiter plate in a dose-dependent manner (FIG. 4A). This binding activity could be removed by preincubation with biotin acrylic beads. In addition, soluble biotin-BSA inhibited the binding of anti-TfR IgG3-C_{H}3-Av to coated plates with 50% inhibition seen at an inhibitor concentration of 0.4 nM (FIG. 4B). Thus, the anti-TtR antibody-avidin fusion protein retains its specificity for rat transferrin receptors and ability to bind to biotin.

Pharmacokinetics, Brain Delivery, and Serum Stability of [³H]-Biotin Bound to Anti-TfR IgG3-C_{H}3-Av.

Rats were injected intravenously with OX-26 (IgG2a anti-TfR) labeled with tritium, or with OX-26 chemically conjugated to Av or anti-TfR IgG3-C_{H}3-Av labeled by incubation with [³H]-biotin and the radioactivity followed for 60 min. (FIG. 5). [³H]-biotin bound to the OX-26/Av chemical conjugate was removed rapidly from the plasma compartment, while the rate of removal of [³H]-biotin bound to anti-TfR IgG3-CH₃-Av is similar to that of [³H] labeled OX-26 (FIG. 5). The corresponding pharmacokinetic parameters obtained by fitting the data to a mono- or bi-exponential equation are given in Table 1. These data show that [³H]-biotin bound to anti-TfR IgG3-C_{H}3-Av is cleared from the peripheral compartment 5.8-fold more slowly than [³H]-biotin bound to the OX-26/Av chemical conjugate. The plasma “area under the plasma concentration curve” (AUC) of [³H]-biotin bound to the anti-TfR IgG3-C_{H}3-Av for the period of 0 to 60 min was increased by a factor of 2.8 compared to that of [³H]-biotin bound to the OX-26/Av conjugate, as a consequence of both a longer half-life of elimination (80.6±4.8 min vs. 20.5±2.2 min) and an increased “mean residence time” (MRT) (114±7 min vs. 16.0±1.3 min). Brain uptake of [³H]-biotin bound to anti-TfR IgG3-C_{H}3-Av was increased by a factor of 6.1 compared to that of the OX-26/Av conjugate (Table 1) reflecting both a 2.6-fold increase in the BBB PS product (2.25±0.65 μL min⁻¹ g⁻¹ vs. 0.85±0.02 μl min⁻¹ g⁻¹) and the higher AUC. These results showed that the fusion protein has much longer serum half-life than the chemical conjugate between OX-26 and avidin, and, most importantly, this fusion protein demonstrated superior [³H]-biotin uptake into brain parenchyma in comparison with the chemical conjugate.

Systemic clearance of [³H]-biotin bound to anti-TfR IgG3-C_{H}3-Av is mainly by the liver, which cleared 5.6±0.7% ID/g within 60 min following an intravenous injection, while its renal clearance is minor with 0.37±0.18% ID/g (Table 2). This means that the binding of [³H]-biotin to anti-TfR IgG3-C_{H}3-Av is very stable in serum. The serum stability of [³H]-biotin/anti-TfR IgG3-C_{H}3-Av fusion protein complex was examined by fast protein liquid chromatography (FPLC) (data not shown). Examination of the FPLC profile indicated that more than 90% of the plasma radioactivity ([³H]-biotin) eluted as the anti-TfR IgG3-C_{H}3-Av complex 60 min after injection with little free [³H]-biotin detected in the serum. These results suggest that it should be possible to use the antibody-avidin fusion protein as a vehicle to deliver biotinylated compounds to the brain.

Brain Uptake of [¹²⁵I]-biotin-PNA Bound to Anti-TfR IgG3-C_{H}3-Av.

Experiments were then performed to determine whether the anti-TfR IgG3-C_{H}3-Av fusion protein can be used to deliver a biotinylated 18-mer antisense specific for the rev gene of HIV-1 (biotin-PNA), a molecule with therapeutic potential against HIV, to the brain. [¹²⁵I]-biotin-PNA was injected intravenously into rats with or without anti-TfR IgG3-C_{H}3-Av and the brain uptake analyzed as described above (Table 3). The brain uptake of unconjugated [¹²⁵I] biotin-PNA was negligible with a PS product of 0.12±0.01 μl min⁻¹ g⁻¹ and a brain uptake of 0.0083±0.0009% ID/g. In contrast, the brain uptake of [¹²⁵I]-biotin-PNA bound to anti-TfR IgG3-C_{H}3-Av was 0.12±0.01% ID/g at 60 min after an intravenous injection and its BBB PS product was 0.67±0.09 μl min⁻¹ g⁻¹. The PS product for the [¹²⁵I]-biotin-PNA was increased 5.6-fold and brain uptake was increased 14.5-fold when the [¹²⁵I]-biotin-PNA was bound to anti-TfR IgG3-C_{H}3-Av. Thus, this novel antibody-avidin fusion protein can deliver the biotinylated antisense drug, anti-HIV PNA, across the blood-brain barrier, suggesting that brain delivery of anti-HIV PNA with the anti-TfR IgG3-C_{H}3-Av may provide an effective treatment for cerebral acquired immune deficiency syndrome (AIDS).

Discussion

Following intravenous injection, biotin bound to Av is rapidly removed from plasma with a half-life of 1.3 min (24). This rapid rate of plasma clearance has been attributed to the attached carbohydrate and the cationic charge of Av, which has 9 lysine and 8 arginine residues, leading to a pI of 10. It is not surprising that chemical conjugation of Av to OX-26 leads to a reduced plasma AUC and a marked reduction of brain targeting compared with OX-26 (Table 1) (29). It was therefore unexpected that genetic fusion of Av to human IgG3 would result in a protein with a half-life similar to that of OX-26. In related studies we have shown that the half-life of anti-TfR IgG3-C_{H}3-Av is similar to that of anti-TfR IgG3.

It is difficult to explain why the antibody chemically conjugated to Av has such different pharmacokinetic properties. The fusion proteins are homogeneous with one Av attached at the end of the heavy chain. The conjugated proteins would be expected to be heterogeneous, varying both in the site and number of attached Av. The IgG-Av fusion protein behaves similar to the IgG-CD4 immunoadhesin, which is an IgG-CD4 fusion protein (30). Free CD4, a cationic protein like Av, is rapidly removed from the bloodstream (30). However, the plasma clearance of CD4 is greatly reduced when the protein is administered in the form of an IgG-CD4 fusion protein (30).

The amount of a drug delivered to the brain is typically expressed as the % ID/g which is a function of the BBB permeability-surface area (PS) product and the plasma AUC (28). The more efficient brain uptake of [³H]-biotin bound to anti-TfR IgG3-C_{H}3-Av (compared to the chemical conjugate) with an accumulation of 0.25% ID/g at 60 min after the intravenous bolus reflect both its improved PS and AUC. This brain concentration is 3-fold higher than the brain uptake after 60 min of the classical neuroactive alkaloid morphine (0.081% ID/g) (28) and is comparable to that of OX-26.

Antisense oligodeoxynucleotides such as anti-HIV PNA may provide an effective therapy for HIV type 1 present in cerebral AIDS. Indeed, antisense oligonucleotides administered by intracerebroventricular injection or infusion have actually demonstrated selective inhibition of in vivo gene expression in the brain (31, 32). However, it would be desirable to have a non-invasive method of administering the oligonucleotides, but unfortunately they show negligible transcellular transport (33). In the present study, the brain uptake of free biotin-PNA (biotinylated anti-HIV PNA) injected intravenously was negligible (0.0083% ID/g). When biotinylated PNA was bound to the OX-26/streptavidin chemical conjugate, the brain uptake of systemically administered biotin-PNA was enhanced to about 0.075% ID/g (23).

However, when anti-TfR IgG3-C_{H}3-Av was used as the delivery vehicle, the brain uptake of biotinylated PNA increased to 0.12% ID/g, a 15-fold increase compared to free biotin-PNA. Thus, the brain uptake of biotin-PNA with the genetically engineered arti-TfR IgG3-C_{H}3-Av is higher than that of biotin-PNA with the OX-26/streptavidin chemical conjugate. Nevertheless, the brain uptake of biotin-PNA bound to anti-TfR IgG3-C_{H}3-Av was half that of biotin bound to anti-TfR IgG3-C_{H}3-Av. The PS product (0.67 μl/min/g brain) of anti-TfR IgG3-C_{H}3-Av/biotin PNA decreased to 30% of the PS product (2.25 μl/min/g brain) of anti-TfR IgG3-C_{H}3-Av/biotin. The decreased brain uptake may reflect the poor intrinsic intracellular permeability of the PNA moiety in the complex.

Our studies have indicated that anti-TfR IgG3-C_{H}3-Av may be able to serve as a universal vehicle for targeting the brain with a vast array of different compounds, including chemicals, proteins, and DNA. In particular, we have demonstrated that anti-TfR IgG3-C_{H}3-Av can enhance the brain uptake of anti-HIV PNA and may provide a treatment for cerebral AIDS. Although we have focused our discussion on targeting the cerebral hemisphere, the anti-TfR IgG3-C_{H}3-Av can also be useful for targeting other structures of the central nervous system, such as the cerebellum and spinal cord, which are also limited by the BBB. Therefore, the results presented here suggest that our novel universal vehicle will have a large number of potential applications in the diagnosis an/or therapy of various central nervous system disorders.

### REFERENCES

The following references are for Example 2.
1. Crone, C., and S. P. Olesen. 1982. Electrical resistance of brain microvascular endothelium. Brain Res. 241:49.
2. Butt, A. M., H. C. Jones, and N. J. Abbott. 1990. Electrical resistance across the blood-brain barrier in anaesthetized rats: a developmental study. J Physiol (Lond). 429:47.
3. Pardridge. W. M. 1991. Peptide Drug Delivery to the Brain, Pardridge. W. M., ed. Raven Press. New York p. 123.
4. Brightman. M. W., and J. H. Tao-Cheng. 1993. Tight Junctions of Brain Endothelium and Epithelium. In The Blood-Brain Barrier: Cellular and Molecular Biology, Pardridge, W. M., ed. Raven Press, New York. p. 107.
5. Abbott, N. J., and I. A. Romero. 1996. Transporting therapeutics across the blood-brain barrier. Mol Wed Today. 2:106.
6. Shapiro, W. R., and J. R. Shapiro. 1986. Principles of brain tumor chemotherapy. Semin Oncol. 13:56.
7. Olson, L., E. O. Backlund, T. Ebendal, R. Freedman, B. Hamberger, P. Hansson, B. Hoffer, U. Lindblom, B. Meyerson, I. Stromberg, and et al. 1991 Intraputaminal infusion of nerve growth factor to support adrenal medullary autografts in Parkinson's disease. One-year follow-up of first clinical trial. Arch Neurol. 48.373.
8. Olson, L., A. Nordberg, H. Vonholst, L. Backman, T. Ebendal, I. Alafuzoff, K. Amberla, P. Hartvig, A. Herlitz, A. Lilja, H. Lundqvist, B. Langstrom, B. Meyerson, A. Persson, M. Viitanen, B. Winblad, and A. Seiger. 1992. Nerve Growth Factor Affects C-11-Nicotine Binding, Blood Flow, Eeg, and Verbal Episodic Memory In an Alzheimer Patient. J Neur TR-P. 4:79
9. Gash, D. M., Z. Zhang, and G. Gerhardt. 1998. Neuroprotective and neurorestorative properties of GDNF. Ann Neurol 44:S121.
10. Duffy, K. R. and W. M. Pardridge. 1987. Blood-brain barrier transcytosis of insulin in developing rabbits. Brain Res. 420:32.
11. Fishmran, J. B. J. B. Rubin, J. V. Handrahan, J. R. Connor, and R. E. Fine. 1987. Receptor-mediated transcytosis of transferrin across the blood-brain barrier. J Neurosci Res. 18:299.
12. Reinhardt R. R., and C. A. Bondy. 1994. Insulin-like growth factors cross the blood-brain barrier. Endocrinology. 135:1753.
13. Shin, S. U., P. Friden, M. Moran, and S. L. Morrison. 1994. Functional properties of antibody insulin-like growth factor fusion proteins. J Biol Chem. 269:4979.
14. Shin, S. U., P. Friden, M. Moran, T. Olson, Y. S. Kang, W. M. Pardridge, and S. L. Morrison. 1995. Transferrin-antibody fusion proteins are effective in brain targeting. Proc Natl Acad Sci USA 92:2820.
15. Friden, P. M., L. K. Walus, G. F. Musso, M. A. Taylor, B. Malfroy, and R. M. Starzyk. 1991. Anti-transferrin receptor antibody and antibody-drug conjugates cross the blood-brain barrier. Proc Natl Acad Sci USA 88:4771
16. Friden, P. M., L. R. Walus, P. Watson, S. R. Doctrow, J. W. Kozarich, C. Backinan, H. Bergman, B. Hoffer, F. Bloom, and A. C. Granholm. 1993. Blood-brain barrier penetration and in vivo activity of an NGF conjugate. Science. 259:373.
17. Walus, L. R., W. M. Pardridge, R. M. Starzyk, and P. M. Friden. 1996. Enhanced uptake of rsCD4 across the rodent and primate blood-brain barrier after conjugation to anti-transferrin receptor antibodies. J Pharmacol Exp Ther. 277:1067.
18. Bullard, D. E., M. Bourdon, and D. D. Bigner. 1984. Comparison of various methods for delivering radiolabeled monoclonal antibody to normal rat brain. J Neurosurg 61:901.
19. Kordower, J. H., V. Charles, R. Bayer, R. T. Bartus, S. Putney, L. R. Walus, and P. M. Friden. 1994. Intravenous administration of a transferrin receptor antibody-nerve growth factor conjugate prevents the degeneration of cholinergic striatal neurons in a model of Huntington disease. Proc Natl Acad Sci USA 91:9077.
20. Granholm, A. C., D. Albeck, C. Backman, M. Curtis, T. Ebendal, P. Friden, M. Henry, B. Hoffer, J. Kordower, G. M. Rose, S. Soderstrom, and R. T. Bartus. 1998. A non-invasive system for delivering neural growth factors across the blood-brain barrier: a review. Rev Neurosci. 9:31.
21. McGrath, J. P., X. Cao, A. Schutz, P. Lynch, T. Ebendal, M. J. Coloma, S. L. Morrison, and S. D. Putney. 1997. Bifunctional fusion between nerve growth factor and a tnansferrin receptor antibody. J Neurosci Res. 47:123.
22. Yoshikawa, T., and W. M. Pardridge. 1992. Biotin Delivery to Brain With a Covalent Conjugate Of Avidin and a Monoclonal Antibody to the Transferrin Receptor J Pharmacol Exp Ther. 263:897.
23. Pardridge, W. M., R. J. Boado, and Y. S. Kang. 1995. Vector-mediated delivery of a polyamide (“peptide”) nucleic acid analogue through the blood-brain barrier in vivo. Proc Natl Acad Sci USA. 92:5592.
24. Shin, S. U., D. Wu, K. Ramanathan, W. M. Pardridge, and S. L. Morrison. 1997. Functional and pharmacokinetic properties of antibody-avidin fusion proteins. J Immunol 158:4797.
25. Jeffries, W. A., M. R. Brandon, A. F. Williams, and S. V. Hunt. 1985 Analysis of lymphopoietic stem cells with a monoclonal antibody to the rat transferrin receptor. Immunology. 54:333.
26. Shin, S. U., and S. L. Morrison. 1989. Production and properties of chimeric antibody molecules. Methods Enzymol. 178:459.
27. Pardridge, W. M. Y. S. Kang, J. L. Buciak. 1994. Transport of human recombinant brain-derived neurotrophic factor (BDNF) through the rat blood-brain barrier in vivo using vector mediated peptide drug delivery. Pharm Res. 11:738.
28. Wu, D., Y. S. Kang, U. Bickel, and W. M. Pardridge. 1997. Blood-brain permeability to morphine-6-glucuronide is markedly reduced compared with morphine. Drug Metab Dispos. 25:768.
29. Kang, Y. S., U. Bickel, and W. M. Pardridge. 1994. Pharmacokinetics and saturable blood-brain barrier transport of biotin bound to a conjugate of avidin and a monoclonal antibody to the transferrin receptor. Drug Metab Dispos. 22:99.
30. Capon, D. J., S. M. Chamow, J. Mordenti, S. A. Marsters, T. Gregory, H. Mitsuya, R. A. Byrn, C. Lucas, F. M. Wurm, J. E. Groopman, and et al. 1989. Designing CD4 immunoadhesins for AIDS therapy. Nature. 337:525.
31. Pollio, G., P. Xue, M. Zanisi, A. Nicolin, and A. Maggi. 1993. Antisense oligonucleotide blocks progesterone-induced lordosis behavior in ovariectomized rats. Brain Res Mol Brain Res. 19:135.
32. Wahlestedt, C., E. M. Pich, G. F. Koob, F. Yee, and M. Heilig. 1993. Modulation of anxiety and neuropeptide Y-Y1 receptors by antisense oligodeoxynucleotides. Science. 259:528.
33 Boado, R. J. 1995. Antisense Drug Delivery Through the Blood-Brain Barrier. Advan Drug Delivery Rev. 15:73.

### EXAMPLE 3

### Use of Antibody Construct of Example 1 to Deliver β-Galactosidase and the Gene Encoding β-Galactosidase into TfR Bearing Cells

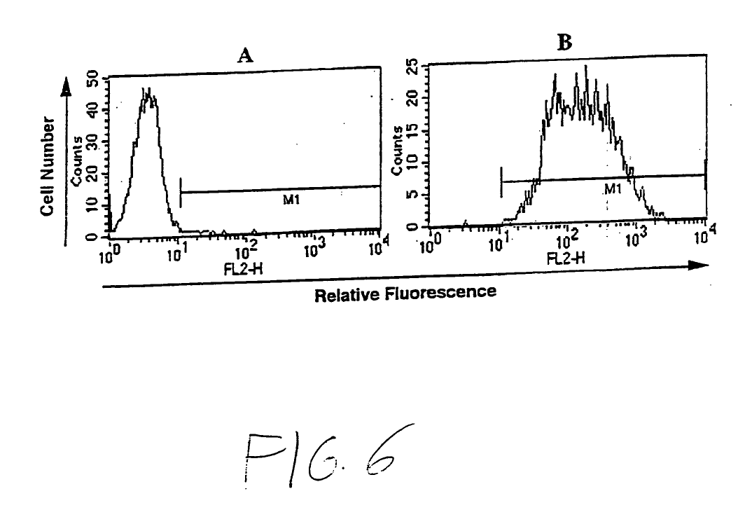

To demonstrate that anti-TfR IgG3-C_{H}3-Av can be used as a universal vector to deliver biotinylated compound into cancer cells that overexpress the TfR w we first studied the ability of anti-TfR IgG3-C_{H}3-Av to target the TfR overexpressed on the surface of cancer cells such as the rat myeloma cell line Y3-Ag1.2.3. FIG. 6 shows flow cytometry demonstrating the specificity of anti-rat TfR IgG3-C_{H}3-Av for TfR: 5×10⁵ rat Y3-Ag1.2.3 cells were incubated with either 1 μg of control anti-DNS IgG3-C_{H}3-Av (panel A) or an anti-rat TfR IgG3-C_{H}3-Av (panel B) for 1 h at 4° C. Then the cells were washed and incubated 1 h at 4° C. with PE-labeled goat anti-human IgG (Pharmingen, San Diego, Calif.) and analyzed by flow cytometry. Analysis was performed with a FACScan (Becton-Dickinson, Mountain View, Calif.) equipped with a blue laser excitation of 15 mW at 488 nm.

Having demonstrated that anti-rat TfR IgG3-C_{H}3-Av binds to the TfR overexpressed on the surface of Y3-Ag_{1.2.3}, the second step was to demonstrate if a complex consisting of anti-rat TfR IgG3-C_{H}3-Av with a biotinylated protein or DNA can be targeted on the surface of the cells. As biotinylated protein we used a commercially available biotinylated β-gal (Sigma, St. Louis, Mo.). As biotinylated DNA we used the expression vector pCH 104 encoding β-gal which we biotinylated using a commercially available reagent (Biotin-Chem-Link, Boehringer Mannheim). For our initial approach we used β-gal and DNA containing more than one biotin per molecule. The optimal molar ratio for the interaction of anti-rat TfR IgG3-C_{H}3-Av and biotinylated β-gal and plasmid was determined by immunoprecipitation and gel retardation electrophoresis (data not shown).

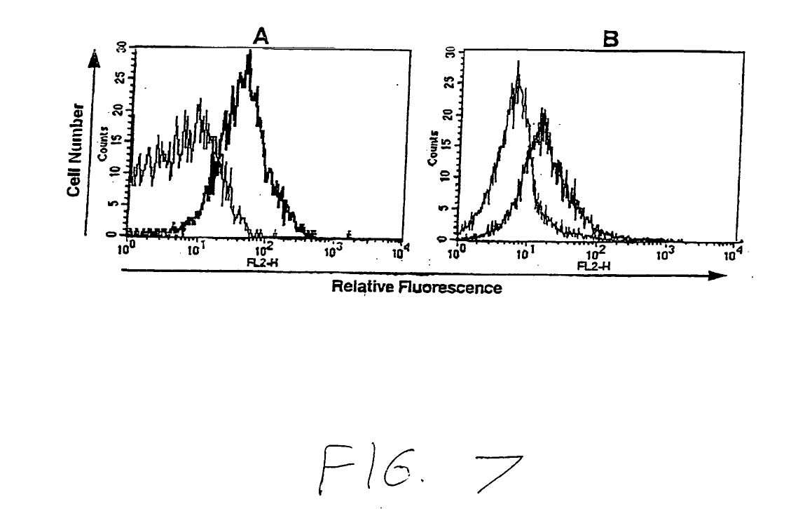

The fact that free anti-rat TfR IgG3-C_{H}3-Av Ab fusion protein binds the TfR on the surface of Y3-Ag1.2.3 does not necessarily mean that a complex consisting of anti-rat TfR IgG3-C_{H}3-Av plus biotinylated molecules of considerable mass such as the β-gal enzyme (464 kDa) and the 12 kb β-gal expression vector would also have the capacity to target the TfR on the surface of Y3-Ag1.2.3. However, if we find that the whole complex (anti-rat TfR IgG3-C_{H}3-Av with a biotinylated protein or DNA) is able to target the cancer cell this will tell us that the Ab fusion protein used as the universal delivery vector (anti-rat TfR IgG3-C_{H}3-Av) is able to keep its antigen target capability after binding the biotinylated enzyme. Localization of DNA or protein to the cell surface would also show that the interaction between the universal vector and the agent it carries is strong enough to keep the complex stable on the surface of the cells even after the binding of the former to the receptor expressed on the surface of the cells. When 1×10⁶ rat Y3-Ag1.2.3 cells were incubated with either 1 μg of anti-rat TfR IgG3-C_{H}3-Av bound to biotinylated β-gal (FIG. 7, panel A, thick solid line) or 1 μg of anti-rat TfR IgG3-C_{H}3-Av bound to biotinylated supercoiled plasmid encoding β-gal (FIG. 7 panel B, thick solid line) for 1 h at 4° C. The molar ratio of universal vector (anti-rat TfR IgG3-C_{H}3-Av) biotinylated compound was 6/1 for biotinylated β-gal and 12/1 for biotinylated DNA. The complex was allowed to form by incubation for 2 h at 4° C. before being added to the cells. Then the cells were washed and incubated 1 h at 4° C. with PE-labeled streptavidin (Pharmingen, San Diego, Calif.) which should bind to free biotin present in the biotinylated compound carried by the antibody fusion protein and analyzed by flow cytometry. As negative isotype specificity control a parallel incubation was done using the same amount of and ratio of conjugate of anti-DNS IgG3-C_{H}3-Av and biotinylated β-gal (panel A, thin solid line) or biotinylated plasmid encoding for β-gal (panel B, thin solid line). Analysis was performed with a FACScan (Becton-Dickinson, Mountain View, Calif.) equipped with a blue laser excitation of 15 mW at 488 nm.

Our next challenge was to prove our hypothesis that after binding to the TfR, the whole complex (vector+biotinylated protein or DNA) will be internalized into the cell by receptor mediated endocytosis and within the cell the protein or DNA will be able to function. To test this hypothesis we decided to use the same reagent that we used to prove the principle that the whole complex (vector+biotinylated protein or DNA) was able to target the surface of the cells. However, we have to first demonstrate that both the β-gal enzyme as well as our expression vector encoding for β-gal did not lose their activities as consequence of biotinylation. The activity of biotinylated β-gal was guaranteed by the supplier (Sigma, St. Louis, Mo.). The β-gal expression vector (pCH 104) was biotinylated at three biotin/DNA ratios (1 biotin/10 bp, 1 biotin/100 bp and 1 biotin/1000 bp) and for standard calcium phosphate transfection. Intracellular β-gal activity was detected by flow cytometry after allowing 48 hours for expression. β-gal activity follows transfection with plasmid with 1 biotin/100 bp and 1 biotin/1000 bp was the same as was obtained using equivalent amount of non-biotinylated DNA (data not shown). However, the activity of the plasmid with 1 biotin/10 bp was significantly lower. A possible explanation for this result is that a large amount of biotin intercalated into the bases of the DNA hampers the transcription machinery. For this reason, we have decided to use a lower level of biotinylation. Using immunoprecipitation and gel retardation electrophoresis we found out that the optimal anti-rat TfR IgG3-C_{H}3-Av/DNA ratio was 12/1 and the optimal level of biotinylation was 1 biotin/100 bp (data not shown) and we have used similar conditions for subsequent experiments. We also found that there are no apparent differences between linear and supercoiled plasmid (data not shown).

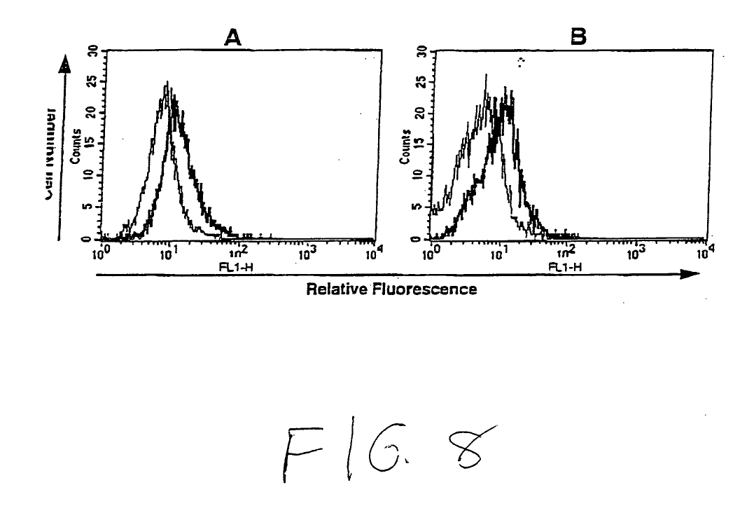

FIG. 8 shows the initial experiment in which the universal vector anti-rat TfR IgG3-C_{H}3-Av was used to deliver biotinylated β-gal enzyme as well as biotinylated plasmid encoding for β-gal (pCH 104) into Y3-Ag1.2.3 cells. 1×10⁶ rat Y3-Ag1.2.3 cells were incubated with either 1 μg of anti-rat TfR IgG3-C_{H}3-Av bound to biotinylated β-gal (panel A, thick solid line) or 1 μg of anti-rat TfR IgG3-C_{H}3-Av bound to biotinylated supercoiled plasmid encoding β-gal (panel B, thick solid line) for 48 h at 37° C. in tissue culture medium. The molar ratio of universal vector (anti-rat TfR IgG3-C_{H}3-Av) biotinylated compound was 6/1 for biotinylated β-gal and 12/1 for biotinylated DNA. The complex was allow to form by incubation for 2 h at 4° C. before being added to the cells. As negative isotype control a parallel incubation was done using a conjugate of anti-DNS IgG3-C_{H}3-Av and biotinylated Egal (panel A, thin solid line) or biotinylated plasmid encoding for β-gal (panel B, thin solid line). The detection of intracellular β-gal activity was made using the DetectaGene™ Green CMFDG lacZ Gene Expression Kit (Molecular Probes Inc, Eugene, Oreg.) which detects by flow cytometry intracellular but not surface associated β-gal. Analysis was performed with a FACScan (Becton-Dickinson, Mountain View, Calif.) equipped with a blue laser excitation of 15 mW at 488 nm.

These experiments demonstrate that anti-rat TfR IgG3-C_{H}3-Av shown in Example 1 can be used to deliver both protein and DNA into cancer cells and importantly, after the

1-83. (canceled)

84. A composition for use in delivering a compound to a cell wherein said cell includes a surface on which an antigen is located, said composition comprising:
a) a fusion protein that is formed by genetically fusing a first segment to a second segment wherein said first segment comprises a variable region of a first antibody that recognizes an antigen located on the surface of said cell wherein said variable region of said antibody undergoes antibody-receptor-mediated endocytosis after binding to said antigen, said first segment further comprising at least one domain of a constant region of a second antibody, and wherein said second segment comprises a protein domain that is genetically fused to said first segment, said protein domain being selected from the group consisting of avidin, an avidin mutein, a chemically modified avidin derivative, streptavidin, a streptavidin mutein and a chemically modified streptavidin derivative; and
b) a conjugated compound that comprises biotin conjugated to a compound to be delivered to said cell, said compound being selected from the group consisting of proteins and nucleic acids said conjugated compound being attached to said fusion protein by a bond between said biotin and said protein domain.

85. A composition according to claim 84 wherein said variable region of said antibody recognizes an antigen that is a receptor.

86. A composition according to claim 85 wherein said receptor is a growth factor receptor.

87. A composition according to claim 86 wherein said receptor is a transferrin receptor.

88. A composition according to claim 84 wherein said conjugated compound comprises a protein.

89. A composition according to claim 88 wherein said protein is an enzyme.

90. A composition according to claim 89 wherein said enzyme is β-galactosidase.

91. A composition according to claim 84 wherein said conjugated compound comprises a nucleic acid.

92. A composition according to claim 91 wherein said nucleic acid is an antisense nucleic acid.

93. A composition according to claim 92 wherein said nucleic acid is antisense to the rev gene of HIV-1.

94. A composition according to claim 93 wherein said conjugated compound is 5′-biotin-CTCCGCTTCTTCCTGCCA-Tyr-Lys-CONH₂-3′.

95. A composition according to claim 91 wherein said nucleic acid is a gene expression vector.

96. A composition according to claim 95 wherein said gene expression vector encodes β-galactosidase.

97. A composition according to claim 91 wherein said nucleic acid is RNA.

98. A composition according to claim 84 wherein said second antibody is a human antibody.

99. A composition according to claim 98 wherein said second antibody is IgG.

100. A composition according to claim 84 wherein said first and second antibodies are not the same.

101. A composition according to claim 100 wherein said first antibody is non-human and said second antibody is human IgG.

102. A composition according to claim 100 wherein said first antibody is an anti-transferrin receptor antibody and said second antibody is human IgG.

103. A composition according to claim 103 wherein said first antibody is OX26 monoclonal antibody.

104. A composition according to claim 84 wherein the constant region of said second antibody consists essentially of a CH1 domain and wherein said second segment is genetically fused to said CH1 domain.

105. A composition according to claim 84 wherein said constant region of said second antibody consists essentially of a CH1 domain and a hinge and wherein said second segment is genetically fused to said hinge.

106. A composition according to claim 84 wherein said constant region of said second antibody consists essentially of a CH1 domain, a hinge, a CH2 domain and a CH3 domain and wherein said second segment is genetically fused to said CH3 domain.

107. A composition according to claim 104 wherein said second antibody is human IgG.

108. A composition according to claim 105 wherein said second antibody is human IgG.

109. A composition according to claim 106 wherein said second antibody is human IgG.

110. A composition according to claim 108 wherein said conjugated compound is 5′-biotin-CTCCGCTTCTTCCTGCCA-Tyr-Lys-CONH₂-3′.

111. A composition according to claim 109 wherein said conjugated compound is 5′-biotin-CTCCGCTTCTTCCTGCCA-Tyr-Lys-CONH₂-3′.


SEQUENCE LISTING (1 sequence)

SEQ ID NO: 1 - DNA, 18 bp, artificial
  18-mer antisense to the rev gene of HIV-1
ctccgcttcttcctgcca